(19) United States
(12) Patent Application Publication
(10) Pub. No.: US 2026/0315241 A1
(43) Pub. Date: Oct. 15, 2026

(54) FLEXURE-BASED INTERBODY IMPLANT

(71) Applicant: NEXUS SPINE, LLC, Salt Lake CIty, UT (US)

(72) Inventors: David T. Hawkes, Salt Lake City, UT (US); Peter A. Halverson, Salt Lake City, UT (US)

(21) Appl. No.: 19/575,689

(22) Filed: Mar. 23, 2026

Related U.S. Application Data
(63) Continuation-in-part of application No. 19/056,478, filed on Feb. 18, 2025.
(60) Provisional application No. 63/556,852, filed on Feb. 22, 2024.
(60) Provisional application No. 63/777,556, filed on Mar. 25, 2025.

(51) Int. Cl. A61F 2/44; A61F 2/30

(52) U.S. Cl. CPC A61F 2/4455; A61F 2/30749; A61F 2002/30014; A61F 2002/30546; A61F 2002/30622

(57) ABSTRACT

Systems and methods for providing a flexure-based standalone interbody fusion device are described. In some implementations, a flexure-based standalone interbody fusion device includes one or more endplates (e.g., a first endplate and a second endplate). In some implementations, the device includes a flexure assembly (e.g., disposed between the two endplates), which in some cases is configured to bear at least a portion of the load applied to the endplates. Some iterations of the flexure assembly include one or more flexures. In some cases, applying a compressive force to the endplates places tension on the flexure, thereby providing a device that is flexible and resistant to buckling and breaking. Additional implementations are described.

### RELATED APPLICATIONS

This application is a continuation-in-part of and claims priority to U.S. patent application Ser. No. 19/056,478, filed Feb. 18, 2025 (Attorney Docket No. 23845.185), and entitled SYSTEMS AND METHODS FOR FLEXURE-BASED STANDALONE INTERBODY FUSION, which claims priority to U.S. Provisional Application Ser. No. 63/556,852 (Attorney Docket No. 23845.178), filed Feb. 22, 2024, and entitled SYSTEMS AND METHODS FOR FLEXURE-BASED STANDALONE INTERBODY FUSION. This application also claims priority to U.S. Provisional Patent Application Ser. No. 63/777,556, filed Mar. 25, 2025 (Attorney Docket No. 23845.222), and entitled FLEXURE-BASED INTERBODY IMPLANT. The contents of each of these applications are incorporated herein by reference in their entirety.

### FIELD

The present disclosure relates to medical implants, and more particularly to spinal implants for interbody fusion.

### BACKGROUND

One significant cause of back pain stems from spinal instability. Chronic spinal instability can lead to modic changes, including bone changes that lead to nerve compression. Nerve decompression is often treated by surgical intervention. In many cases, spinal surgery to decompress nerves (such as disc replacement surgery) requires the removal of stabilizing structures, such as the anterior longitudinal ligament, the posterior longitudinal ligament, and the annulus. Thus, attempts to relieve back pain (or correct other spinal issues) can lead to increased spinal instability and increased pain.

Interbody fusion devices have been developed to restore spinal stability by providing a rigid connection between adjacent vertebral bodies. However, if a spinal segment is made too rigid, it can result in deleterious hypermobility at adjacent spinal segments, and thus cause adjacent level degeneration. Furthermore, immediately after implantation there is often significant motion at the bone-to-implant interface, which can be painful and can inhibit boney attachment. Ultimately, stability at the operative spinal level can rely on relatively slow formation of fibrous tissue in the bone-to-implant interface; after which hypermobility can be caused at the adjacent levels.

In an attempt to counteract some of these issues, such as to prevent adjacent level iatrogenic hyper-mobility, and subsequent adjacent level degeneration, disc replacement devices have been created. Some disc replacement devices provide motion at the operative level and in turn avoid hypermobility at the adjacent levels. However, some existing disc replacement devices are not sufficiently stable and inappropriately recruit surrounding tissues to provide spinal stability. This is often exacerbated by surgically overstuffing the disc space with an implant that is too tall and thus stretching the surrounding tissues beyond their healthy limits to attempt to restrain motion. Accordingly, this can place undue strain on soft tissues, which can damage the tissues (e.g., through excessive stretching). The result can be heterotopic ossification (e.g., bone generation outside of the skeleton), with bone bridging across the disc space, outside the disc space.

Many disc replacement devices rely on sliding components. For example, some devices rely on a ball-in-socket mechanism. Some sliding mechanisms are not inherently stable and do not effectively restore spinal stability. Also, some sliding components generate wear debris (e.g., material grinding or flaking off due to the friction forces generated by sliding over time). The debris from the frictional forces can cause osteolysis (bone degradation) and concomitant problems. In some cases, such devices are encased within a sheath to prevent release of wear debris, but some such sheaths are themselves susceptible to degradation, which can result in bulk release of wear debris upon rupture of the sheath.

Some disc replacement devices rely on polymeric cores to provide stable motion. Polymers can be flexible. However, existing polymeric devices do not appropriately restore stability in all planes of motion. For example, the polymer might provide appropriate flexion/extension stiffness but consequently does not provide appropriate stiffness in lateral bending, axial rotation, nor axial compression. Also, polymers oxidize within the body which changes the stiffness of the polymer. Furthermore, polymers have finite fatigue life and will ultimately fail from wear.

Some devices have decreased rigidity in order to generate additional flexibility, but this can lead to buckling, bending, fatigue, and other undesirable effects on the device. In some devices—in an attempt to make interbody devices that are flexible—yet rigid enough to prevent buckling—carcinogenic materials have been incorporated, which can lead to even more severe medical problems (e.g., cancer) down the road. Moreover, due to their design, many devices are incapable of achieving a desired level of flexibility while still having a desired level of stability, regardless of the type of material used.

Thus, while techniques currently exist that are used to provide stabilizing spinal devices, challenges still exist, including those listed above. Accordingly, it would be an improvement in the art to augment or even replace current techniques with other techniques.

### SUMMARY

It is often desirable for an interbody device (i.e., an interbody fusion device for standalone interbody fusion) to have a load-bearing component that is sufficiently rigid so as to stabilize the spine and to provide the structural support needed to allow the spine to heal and to minimize pain. However, it is also generally desirable for the device to provide enough flexibility on one or more axes, including a flexion/extension axis, a lateral bending axis, and a torsion axis (also called an axial rotation axis) to prevent overloading adjacent spinal levels. If the device is not sufficiently flexible, the spinal levels adjacent to the treated level can often be forced into hypermobility, which can lead to accelerated degeneration, or otherwise cause pain and damage to the patient (which can be long-term or even permanent). Some implementations of the present systems and methods provide an interbody device that is both stable and flexible.

According to some implementations, the interbody device includes one or more endplates (e.g., a first endplate and a second endplate) configured to be disposed adjacent to the natural bony endplates of spinal vertebrae. Additionally, some implementations include one or more flexure assemblies disposed between the endplates and configured to provide stability to the implant when a load is placed on the endplates (e.g., the compressive force applied by the vertebrae between which the implant is disposed). Moreover, some implementations of the flexure assembly are also configured to be flexible (e.g., to mimic one or more physiological conditions of a healthy spine).

In some iterations of the systems and methods disclosed herein, the desired stability and flexibility of the flexure assembly is achieved through the provision of one or more tension-based components, or flexures. Indeed, by providing a tension-based load-bearing component as opposed to a compression-based one, a load can be supported and flexibility can be maintained with a greatly decreased risk of components buckling, fatiguing, or breaking. Accordingly, the described flexure can include any suitable component capable of being placed under tension, and the flexure assembly can include any suitable components (in any suitable configuration) for taking the compressive force applied to the implant endplates and transposing it to a tensile force in the flexure.

In some implementations, the flexure assembly includes one or more loading regions (e.g., for receiving the compressive force) and one or more supports (e.g., for transferring the load from the loading regions to the flexure). For example, a first support can transfer a first portion of a compressive force from a first loading region to a first end of the flexure, and a second support can transfer a second portion of the compressive force from a second loading region to a second end of the flexure. In some cases, the first end of the flexure is positioned opposite to the first loading region, and the second end of the flexure is positioned opposite to the second loading region (e.g., the first end of the flexure is farther from the first loading region than is the second end of the flexure, and the second end of the flexure is farther from the second loading region than is the second end of the flexure). This can allow the flexure (or any suitable portion thereof) to be placed under tension rather than under compression, thus allowing it to have a slighter aspect ratio and better flexibility and stability properties than some compression-based components.

Although in some cases a single flexure assembly is sufficient, some implementations include multiple flexure assemblies. In some implementations, multiple flexure assemblies with specific flexibility profiles have certain orientations allowing them to sum and work together to provide the desired characteristics of the implant as a whole (e.g., specific flexibilities along the flexion/extension axis, the lateral bending axis, and the torsion axis). As many different combinations are possible, additional details are discussed below.

### DESCRIPTION OF THE FIGURES

The objects and features of the present disclosure will become more fully apparent from the following description and appended claims, taken in conjunction with the accompanying figures. Understanding that these figures depict only typical embodiments of the disclosed systems and methods and are, therefore, not to be considered limiting of its scope, the systems and methods will be described and explained with additional specificity and detail through the use of the accompanying figures in which:

FIG. 1 shows a perspective view of an interbody implant, as known in the art;

FIG. 2 shows a perspective view of a flexure-based interbody implant, in accordance with some embodiments of the disclosed systems and methods;

FIG. 3 shows a perspective view of a stem assembly, in accordance with some embodiments;

FIG. 4 shows a perspective view of a flexure assembly, in accordance with some embodiments;

FIG. 5 shows a perspective view of the flexure assembly, in accordance with some embodiments;

FIGS. 6A-6D show perspective views of a plurality of flexure assemblies coupled together in differing configurations, in accordance with some embodiments;

FIG. 7 shows a perspective view of the flexure assembly, in accordance with some embodiments;

FIG. 8 shows a plan view of the flexure assembly, in accordance with some embodiments;

FIG. 9 shows a top perspective view of the flexure-based interbody implant, in accordance with some embodiments;

FIG. 10 shows a plan view of the flexure-based interbody implant, in accordance with some embodiments;

FIG. 11 shows a front elevation view of the flexure-based interbody implant, in accordance with some embodiments;

FIG. 12 shows a side elevation view of the flexure-based interbody implant, in accordance with some embodiments;

FIG. 13 shows a perspective view of the flexure assembly arrangement for the flexure-based interbody implant, in accordance with some embodiments;

FIG. 14 shows a perspective view of the flexure-based interbody implant with a porous structure, in accordance with some embodiments;

FIG. 15 shows a cutaway view of the flexure-based interbody implant with the porous structure, in accordance with some embodiments;

FIG. 16 shows a perspective view of the flexure-based interbody implant with locking caps disposed in secondary fixation passages, in accordance with some embodiments;

FIG. 17 shows a front elevation view of the flexure-based interbody implant installed in an interbody space between two spinal vertebrae, in accordance with some embodiments;

FIG. 18 shows a front perspective view of the flexure-based interbody implant, in accordance with some embodiments;

FIG. 19 shows a front perspective view of the flexure-based interbody implant, in accordance with some embodiments;

FIG. 20A shows a front elevation view of the flexure-based interbody implant, in accordance with some embodiments;

FIG. 20B shows a side elevation view of the flexure-based interbody implant, in accordance with some embodiments;

FIG. 21A shows a top perspective view of the flexure-based interbody implant, in accordance with some embodiments;

FIG. 21B shows a top perspective cutaway view of the flexure-based interbody implant, in accordance with some embodiments;

FIG. 21C shows a plan view of the flexure-based interbody implant, in accordance with some embodiments;

FIG. 22A shows a side perspective view of the flexure-based interbody implant, in accordance with some embodiments;

FIG. 22B shows a side perspective cutaway view of the flexure-based interbody implant, in accordance with some embodiments;

FIG. 23A shows a first elevation view of the flexure-based interbody implant in accordance with some embodiments; and

FIG. 23B shows a second elevation view of the flexure-based interbody implant in accordance with some embodiments.

### DETAILED DESCRIPTION

A description of embodiments will now be given with reference to the figures. It is expected that the present systems and methods may take many other forms and shapes. Hence, the following disclosure is intended to be illustrative and not limiting, and the scope of the disclosure should be determined by reference to the appended claims.

Systems and methods for providing a standalone interbody device are disclosed. Generally speaking, it is often desirable for an interbody device to have a load-bearing component that is sufficiently rigid so as to stabilize the spine and to provide the structural support needed to allow the spine to heal and to minimize pain. However, it is also generally desirable for the device to provide flexibility on or more axes, including a flexion/extension axis, a lateral bending axis, a torsion axis (also called an axial rotation axis), and any combination thereof. Indeed, in some embodiments, it is desirable for the device to provide flexibility in each of the flexion/extension axis, the lateral bending axis, and the torsion axis. If the device is not sufficiently flexible, this can (in some cases) overload adjacent spinal levels, limit a patient's mobility, cause friction between spinal vertebrae and the device, and cause pain and damage to the patient (which can be long-term or even permanent). Thus, some embodiments of the present systems and methods provide an interbody device that is both stable and flexible.

Some implant makers have attempted to provide a stable and flexible device by including one or more sliding components, but such components are not always stable and often generate a substantial amount of wear debris (e.g., from material flaking or rubbing off due to the friction forces generated by sliding over time). The debris and the frictional forces can cause osteolysis (or bone degradation) and other major problems. In some cases, such interbody devices are encased within a sheath to prevent release of wear debris, but some such sheaths are themselves susceptible to degradation, which can result in bulk release of wear debris upon rupture of the sheath. Moreover, sliding components can wear out over time, thereby requiring eventual replacement. Some embodiments of the present systems and methods provide an interbody device that does not have sliding components, thereby averting the aforementioned problems.

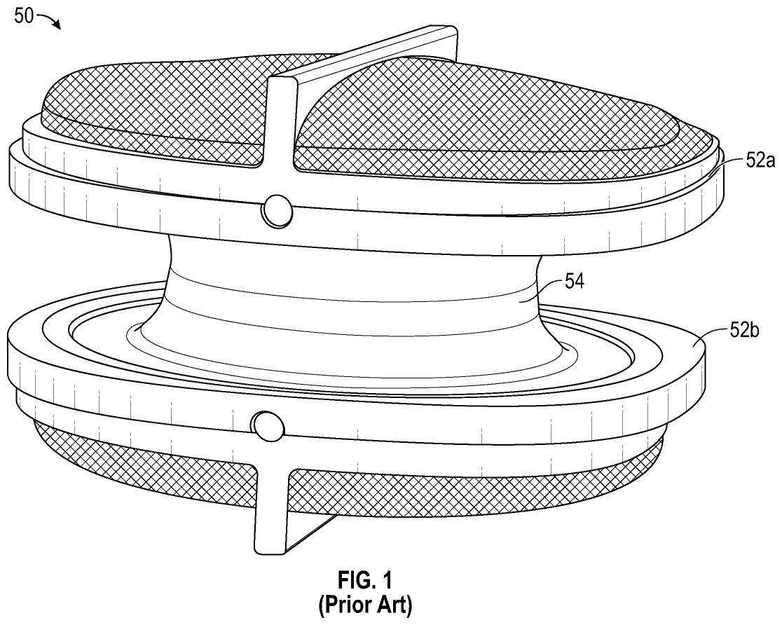

Some interbody devices attempt to provide flexibility without sliding components by utilizing a compression-based flexible member sandwiched between contact structures. For example, FIG. 1 shows an interbody device 50 having a first contact structure 52a and a second contact structure 52b, with a stem 54 spanning the gap between the two contact structures, as taught in the prior art. This configuration can have several substantial drawbacks. For one, the stability of the device can increase proportionally to the thickness of the stem, but the flexibility of the device can decrease proportionally to the thickness of the stem. Accordingly, stability and flexibility can have an inversely proportional relationship in such devices (and cannot, generally, both be increased together). Thus, when the stem is thin enough to be sufficiently flexible, it can also be weaker, and therefore it can be highly susceptible to fatigue failure, buckling and breaking. Furthermore, some compression-based flexible structures do not provide anatomical stiffness simultaneously in all planes of motion. For example, a human cervical disc is relatively incompressible but quite flexible in flexion/extension, whereas a helical coil spring is often a compression-based flexible structure that has a compression stiffness directly related to its flexion/extension stiffness.

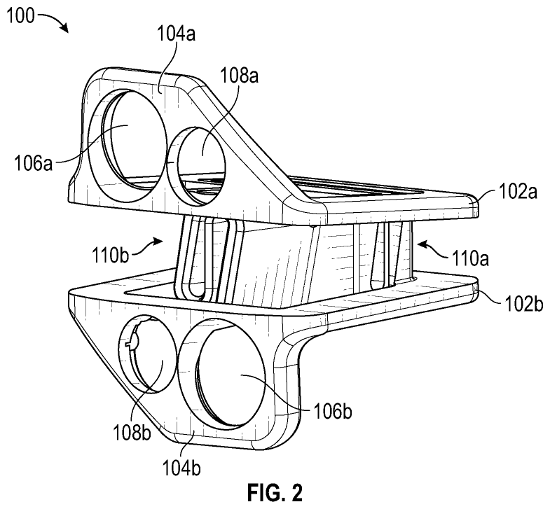

Some embodiments of the present systems and methods address one or more of the foregoing issues by providing a flexure-based standalone interbody implant 100. The implant can include any component suitable for use with an interbody device, and in particular an interbody device that has one or more flexure assemblies 110 for providing stability and flexibility to the implant. For example, FIG. 2 shows an embodiment of a flexure-based interbody implant 100 having a first endplate 102a and a second endplate 102b (each optionally having a flange 104a/104b with a primary fixation passage 106a/106b and a secondary fixation passage 108a/108b formed therethrough), with the first endplate 102a being separated from the second endplate 102b by one or more flexure assemblies (e.g., a first flexure assembly 110a and a second flexure assembly 110b), as opposed to using a single stem (such as the stem 54 of FIG. 1).

Using one or more flexure assemblies 110 (with one or more tension-based flexures 114 as opposed to a single compression-based stem 54 operating as a load-bearing component) can provide one or more features, so various flexure assembly configurations—as well as other implant elements utilized in some embodiments—are discussed below. In some cases, this includes providing flexure geometry that can restore appropriate stability in each and every plane of spinal motion (e.g. flexion/extension, lateral bending, axial rotation, and compression). The ranges of stiffness for each plane of spinal motion can be unique to that plane, and can vary for each level of the spine. Independently and simultaneously matching each stiffness, is a feature of some embodiments of the described flexure geometry that is not found in most, if any, competing devices. Moreover, a flexible interbody device that specifically and simultaneously matches stiffness for each plane (or specific planes) of spinal motion and then also has a graft window (as provided by some embodiments described herein) is also a feature that is not found in most, if any, competing devices.

The described implant 100 can include any suitable component or characteristic that allows it to function as described herein. Indeed, in some embodiments, the implant includes one or more endplates 102 (such as a first endplate 102a and a second endplate 102b). The endplate can include any suitable component configured to be placed adjacent to, align with, couple with, or otherwise interact with the natural bony endplate of a patient's spinal segment. For example, the endplates can include one or more plates, sheets, frames, surfaces, planes, load-bearing regions, scaffolds, surfaces, contacts, porous surfaces, or other components configured to act as an artificial endplate or to interface with a natural bony endplate. Additionally, the endplates can have any suitable configuration that allows them to function as intended. Indeed, in some embodiments, one or both of the endplates is planar, substantially planar, domed, convex, concave, symmetrical, asymmetrical, undulated, texturized, smooth, shaped to define one or more recesses, shaped to include one or more projecting surfaces, configured to include one or more surfaces that are configured to conform to a corresponding bone contact surface, or otherwise has any other suitable shape or feature. By way of non-limiting illustration, FIGS. 2, 9, 12, 14, and 16 show some embodiments of implants 100 that each have a first endplate 102a and a second endplate 102b, wherein each of the endplates include a substantially planar plate or frame configured to be disposed adjacent to a natural bony endplate of a spinal segment. Generally, bio-compatible materials that have been made rough provide suitable materials for boney ingrowth.

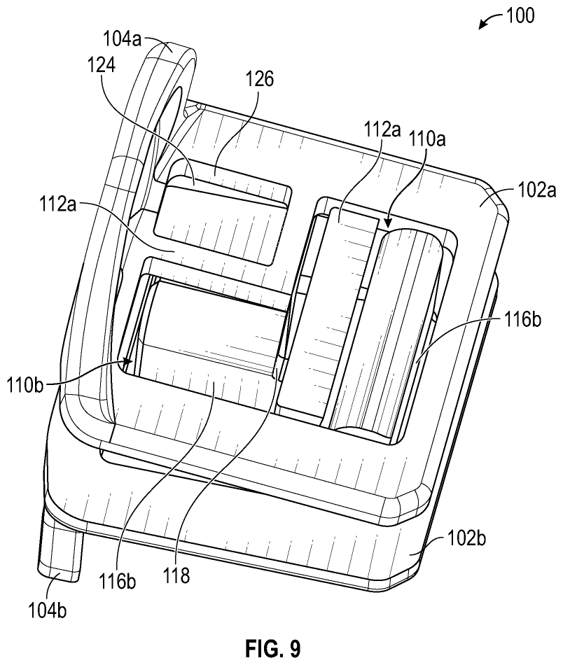

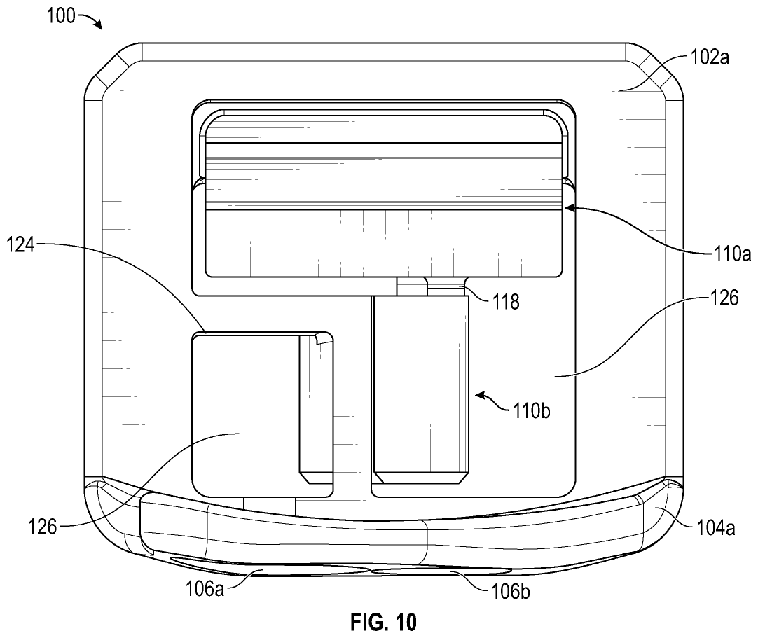

The endplate 102 can have any shape suitable for interfacing with a natural endplate. For example, some embodiments of the endplate are (or are substantially) circular, semi-circular, triangular, square, rectangular, trapezoidal, pentagonal, hexagonal, star-shaped, T-shaped, polygonal, rounded, symmetrical, asymmetrical, shaped to match or otherwise correspond with a natural endplate, or any other regular or irregular shape capable of interfacing with a natural endplate. By way of non-limiting illustration, FIGS. 9-10 show some embodiments in which the endplates 102a and 102b are substantially rectangular or quadrilateral in shape. Generally, maximizing contact area can minimize contact stress to provide for bone health. Also, filling the intervertebral space to the perimeter of each endplate can help provide for contacting the strongest portion of the vertebral endplate.

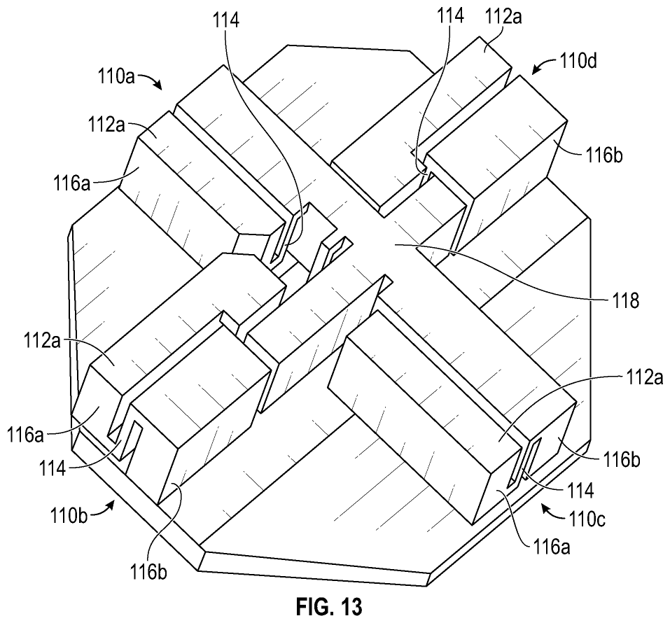

Some embodiments of the endplate 102 are solid or unbroken, while others are broken or discontinuous. Moreover, some embodiments are smooth, while others are knurled, porous, perforated, roughened, undulated, recessed, provided with one or more protrusions, or are otherwise textured. Indeed, some embodiments of one or more endplates are flat, while others are contoured. By way of non-limiting illustration, FIGS. 2, 9, 12, 14, and 16 show some embodiments of implants 100 that each have a first endplate 102a and a second endplate 102b that each include a substantially planar plate or frame with one or more gaps (e.g., windows, as discussed in more detail below) formed therein.

Where the described implant 100 includes multiple endplates 102, the endplates can run parallel or substantially parallel with respect to each other when the implant is at rest, or they can be angled with respect to each other when the implant is at rest. By way of example, FIG. 12 shows an embodiment of the implant 100 with its first endplate 102a and its second endplate 102b being slightly angled such that they are slightly farther apart at a ventral side (near flanges 104a and 104b) than at a dorsal side.

According to some embodiments, the implant 100 includes one or more primary fixation mechanisms. The primary fixation mechanism can include any component configured to secure the implant in the proper location within the patient. For example, the primary fixation mechanism can include any component configured to secure the implant in an intervertebral space (e.g., by securing it to one or more spinal vertebrae).

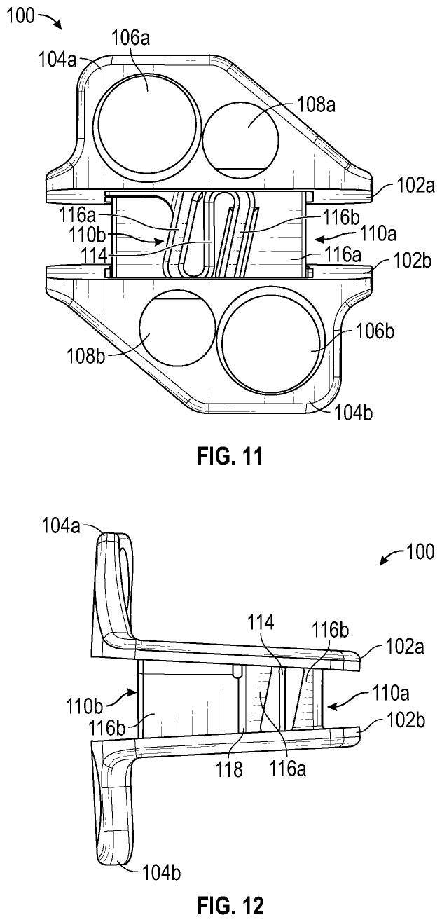

In some embodiments, the primary fixation mechanism includes one or more anchors, catches, flanges, fasteners, burrs, barbs, frictional engagements, mechanical engagements, and any other suitable feature that can be used to secure the implant 100 in an intervertebral space. Indeed, in some embodiments, the primary fixation mechanism comprises one or more flanges 104.

Where the primary fixation mechanism includes a flange, the flange can include any suitable component that is configured to receive one or more anchors 107 or to couple to a portion of a patient (e.g., to a spinal vertebra). The flange can also be any suitable shape, including being substantially semi-circular, square, rectangular, triangular, trapezoidal, pentagonal, hexagonal, otherwise polygonal, symmetrical, asymmetrical, or any other regular or irregular shape suitable for receiving an anchor. By way of non-limiting illustration, FIG. 11 shows an embodiment, in which the flange 104a is somewhat quadrilateral and asymmetrical in shape.

The flange 104 can also be coupled to the endplate 102 or any other portion of the implant 100 in any suitable manner, such as via one or more nails, screws, bolts, staples, eyelets, adhesives, welds, interference fits, friction fits, tongue-and-groove connections, snaps, ties, rivets, or any other coupling mechanism, or by being integrally formed with one or more components of the implant. Moreover, the flange can be coupled at any angle, including parallel, perpendicular, acute, obtuse, at an intersecting angle with respect to a corresponding endplate, or any other suitable configuration or combination thereof. By way of non-limiting illustration, FIG. 11 shows an embodiment of the implant 100 with a first flange 104a and a second flange 104b, with the first flange being integrally formed with a first endplate 102a, and the second flange being integrally formed with a second endplate 102b, with each flange having a somewhat of a rounded, polygonal shape with a substantially rectangular or quadrilateral portion in which one or more primary fixation passages 106a and 106b are formed, and a substantially triangular portion or sloped portion in which one or more secondary fixation passages 108a and 108b are formed. In FIG. 11, each flange is substantially perpendicular to its respective endplate, and is coupled to each respective endplate at a ventral edge of the endplate.

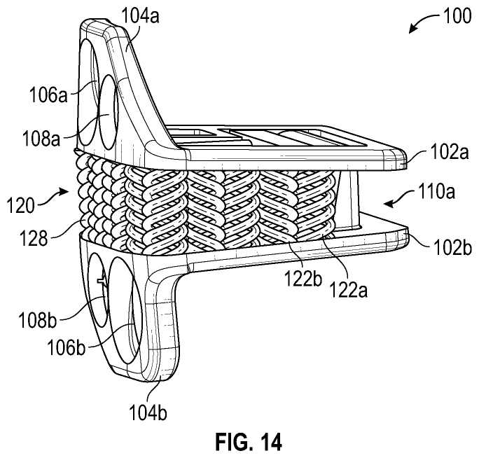

As briefly mentioned in the example in the preceding paragraph, some embodiments of the implant 100 include one or more primary fixation passages 106. The primary fixation passage can include any suitable feature that it is configured to couple with one or more anchors 107. Moreover, some embodiments include one or more secondary fixation passages 108. The secondary fixation passage can include any feature that it is configured to couple to one or more secondary anchors or any other suitable component for further securing the anchor 107 in place, such as a locking cap 109 that is configured to selectively lock a primary anchor in place. By way of non-limiting illustration, FIG. 11 shows an embodiment of the implant 100 with flanges 104a and 104b, each of which has a primary fixation passage 106a and 106b and a secondary fixation passage 108a and 108b. In FIG. 11, the primary fixation passages 106 are substantially circular, and are formed in a somewhat rectangular portion of each of the respective flanges 104. The secondary fixation passages 108 in FIG. 11 are also substantially circular, are optionally smaller than the primary fixation passages 106, and are formed in a sloped, angled, or tapered portion of the respective flanges 104. Similar passages 106 and 108 can also been seen in the implant 100 of FIG. 14.

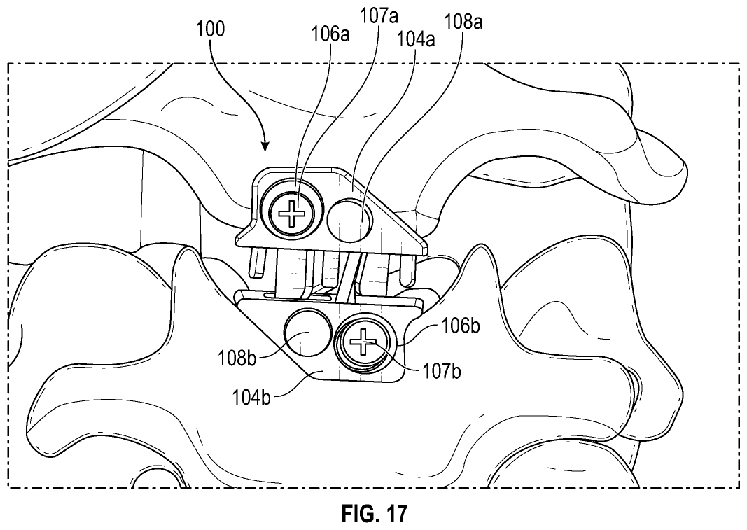

As mentioned, some embodiments of the implant 100 include one or more anchors 107. Where the implant includes an anchor, the anchor can include any suitable anchoring component for securing the implant in place, such as one or more screws, bolts, threaded couplings, nails, stakes, pins, staples, pawls, spikes, blades, barbs, catches, or any other suitable anchors. By way of non-limiting illustration, FIG. 17 shows an embodiment of the implant 100 with a first anchor 107a in the form of a bolt or screw placed through a first primary fixation passage 106a, and a second anchor 107b in the form of a bolt or screw placed through a second primary fixation passage 106b, thereby securing the implant to a patient's spinal vertebrae.

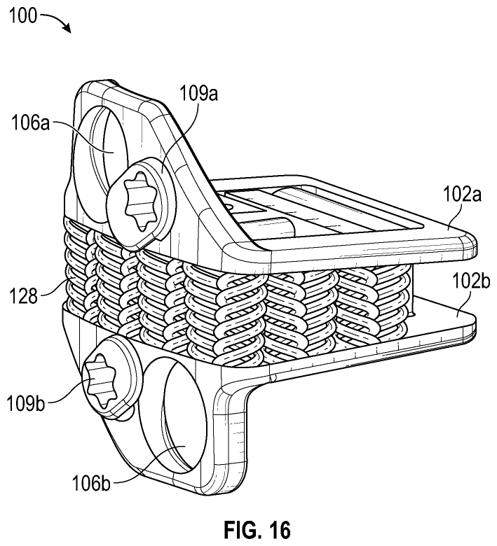

Some embodiments of the implant 100 include one or more secondary securement components. Where the implant includes a secondary securement component, the secondary securement component can include anything that helps the anchors 107 remain fixed in place, such as one or more secondary anchors, locks, caps, or other securement components. For example, some embodiments include one or more locking caps 109 configured to selectively cover at least a portion of a head of the anchor to prevent the anchor from backing out. Where the implant includes a locking cap, the locking cap can have any component or configuration that allows it to help prevent an anchor from backing out. For example, in some embodiments the locking cap has a lip or protection that is configured to selectively cover all or a portion of the anchor (e.g., by covering all or a portion of the primary fixation passage 106). In some embodiments, the locking cap is rotatable with respect to the secondary fixation passage 108 such that the lip can be rotatably disposed in front of the primary fixation passage (thereby blocking an anchor from being inserted or removed therethrough), or rotatably disposed in an unlocked position wherein the lip does not cover the primary fixation passage, thereby allowing an anchor to be inserted or removed. In some embodiments, the locking cap is configured to lock in place in the deployed position (e.g., by snapping, hooking, engaging with a locking component, or otherwise locking in place). By way of non-limiting illustration, FIG. 16 shows an embodiment of the implant 100 with a first locking cap 109a placed through a first secondary fixation passage 108a and a second locking cap 109b placed through a second secondary fixation passage 108b, wherein the locking caps are configured to be rotated to cover a portion of the respective primary fixation passages (and thereby prevent anchors from backing out of said primary fixation passages).

Regarding general characteristics of the implant 100, some embodiments of the implant provide a desired range of flexibility along one or more of a flexion/extension axis, a lateral bending axis, and an axial rotation or torsion axis. Though it does not necessarily occur in all embodiments, in some embodiments, the amount of flexibility in each of these planes matches that of the adjacent healthy spinal levels so as to restore stability without making the segment too rigid. In some embodiments, the desired range of flexibility closely matches the physiological range (although the flexibility of the implant can also be greater or lesser than physiological flexibility, if desired). In some embodiments, the desired range of flexibility depends on the target level of the spine (or other area of the body in which the implant will be used), and it may vary between spinal segments of a patient or across patients. In accordance with some embodiments, the desired degree of flexibility can be anywhere between 0 degrees and 20 degrees, or any subrange thereof (e.g., between 1 and 15 degrees, between 2 and 10 degrees, or any other desirable subrange). By way of non-limiting illustration, in some embodiments the flexion/extension flexibility of the implant is 8 degrees±3 degrees for cervical spine implementations, or 6 degrees±3 degrees for lumbar spine implementations (or 7 degrees±4 degrees generally). In some embodiments, the lateral bending flexibility of the implant is 6 degrees±2 degrees for cervical spine implementations, or 4 degrees±2 degrees for lumbar spine implementations (or 5 degrees±3 degrees generally). In some embodiments, the axial rotation flexibility is 5 degrees±3 degrees for cervical spine implementations, or 2 degrees±2 degrees for lumbar spine implementations (or 4 degrees±3.5 degrees generally). In some embodiments, the range of motion for a particular spinal segment of a particular patient is measured prior to operation (e.g., using preoperative X-rays, MRI, CT scan, or any other suitable imaging technique) and the flexibility of the implant is tailored to match the measured levels of motion (or to simulate ideal levels of motion). For example, any of the foregoing values can be adjusted by an amount between 0.1 degrees and 2 degrees, or any subrange thereof (e.g., between any two of the following: 0.1, 0.2, 0.3, 0.4, 0.5, 0.6, 0.7, 0.8, 0.9, 1.0, 1.25, 1.5, 1.75, 2, or any other value that is less than two degrees in order to achieve the desired levels of flexibility).

In some embodiments, the implant 100 has a certain compression stiffness while having a different, lower stiffness in bending. The compression stiffness can be any stiffness suitable for a medical implant, such as any stiffness desirable for insertion between two adjacent vertebrae of a patient's spine and is, in some embodiments, made higher than the minimum compression stiffness of an intervertebral disc. Though the implant can have any suitable bending stiffness, in some embodiments, the bending stiffness is less stiff than the compression stiffness and is stiff enough to restore equal motion to the adjacent, healthy levels. Specifically, in some embodiments, the compression stiffness of the device is at or above the compressive stiffness of an intervertebral disc while the bending stiffness is at (or similar to) the bending stiffness of a healthy intervertebral disc, which can be measured in flexion/extension with X-rays of the adjacent spinal levels. In some cases, being relatively stiff in compression while being less stiff in flexion/extension is feature that differentiates the described systems and methods from most, if not, all other competing devices. Furthermore, it is believed that having a different stiffness in each plane of bending is a feature of some embodiments of the described systems and methods that is not found in most, if any, competing devices, noting that such feature can allow the operative level to be restored to healthy levels in each plane of motion. In this regard, some competing devices have been too flexible in flexion/extension or too flexible in axial compression or too flexible in axial rotation, etc. On this point, getting all of the stiffnesses right, at the same time, is a feature of some embodiments of the described implant. In some embodiments, the compression stiffness of the implant (or a portion thereof) is between 100 MPa and 120 GPa (or any subrange thereof)—allowing the implant to protect exiting nerve roots from being compressed and noting that approximately 120 GPa is the stiffness of solid titanium, which is a desired material for some embodiments of this implant.

In some embodiments, the bending stiffness of the implant 100 is closer to the range of between 20 deg/N-M and 0.25 deg/N-M (or any subrange thereof, such as 4±2 deg/N-M or any other suitable subrange). For example, some embodiments of the implant have a compressive stiffness of between: 100 MPa and 2 GPa, 200 MPa and 1.5 GPa, 300 MPa and 1.2 GPa, 500 MPa and 850 MPa, or any other subrange between 100 MPa and 120 GPa, while simultaneously having a bending stiffness of between: 0.25 deg/N-M and 5 deg/N-M, 3 deg/N-M and 5 deg/N-M, 1 deg/N-M and 8 deg/N-M, or any other subrange between 0.25 deg/N-M and 20 deg/N-M. According to some embodiments, the stiffness is sufficient for the implant (or one or more load-bearing components of the implant, as discussed below) to withstand a certain compressive force (e.g., the compressive force applied by the adjacent vertebrae of a patient's spine when the implant is inserted between such vertebrae) without compression of exiting nerve roots and while still allowing stable flexion/extension, lateral bending, and axial rotation at (or near) physiologic loads.

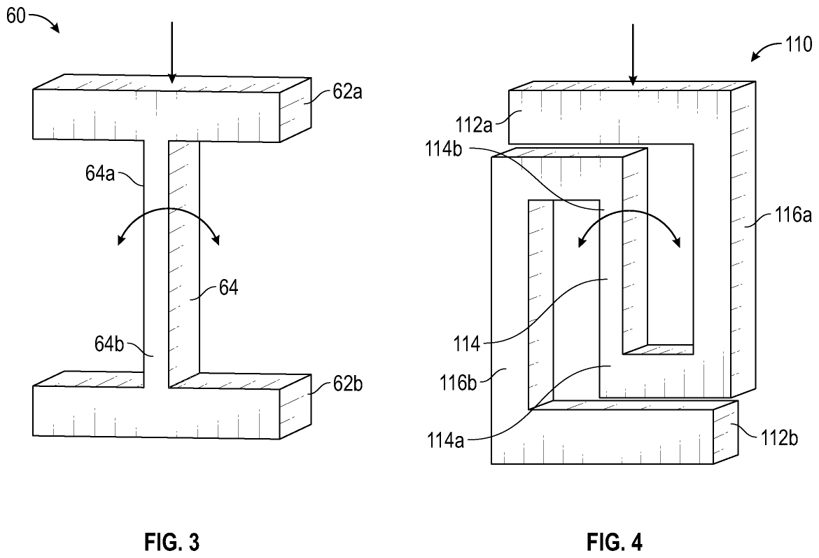

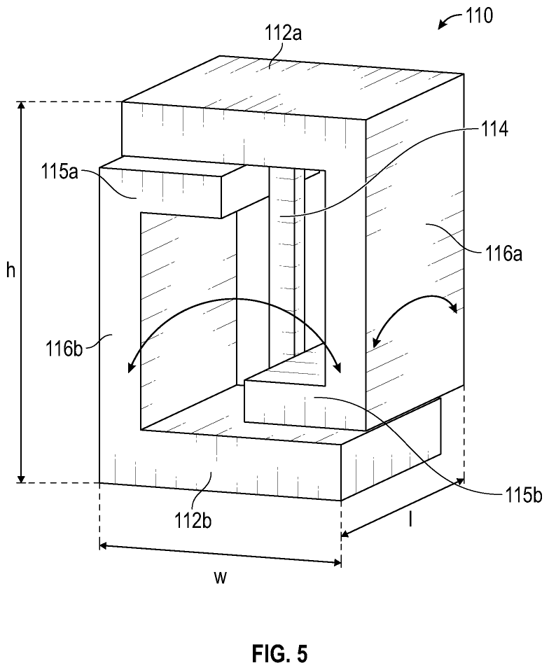

To help ensure that desired flexibility and stability parameters can be achieved, some embodiments of the implant 100 include one or more flexure assemblies 110. Generally speaking, some compression-based flexible elements are unstable, whereas some tension based flexible elements can be substantially more stable. To illustrate, an object suspended from a rope hanging from the ceiling is (in some cases) much less likely to fall than an object balanced on a pole standing on its end on the floor. Moreover, in many cases, it is a lot easier to bend or break a rod by compressing it from its ends than to bend or break the rod by pulling on its ends directly outward. By way of example, FIG. 3 shows a stem assembly 60 having one or more load-bearing regions 62a/62b separated by one or more stems 64. When pressure is exerted on one or more of the load-bearing regions 62, the stem is liable to fail in fatigue, buckle, collapse, or break (especially when undergoing flexing along one or more axes). In contrast, FIG. 4 shows an embodiment of a flexure assembly 110 having one or more load bearing regions 112a/112b separated by one or more tension-based flexures 114. In this embodiment, the flexure 114 is supported by a first support 116a and a second support 116b. When pressure is exerted on one or more of the load-bearing regions, the flexure 114 is placed under tension and is not likely to bend and break, even if it undergoes significant flexing along any or all of the flexion/extension axis, lateral bending axis, or torsion axis. Moreover, as shown in FIG. 5, the stability of the flexure assembly 110 can, in some embodiments, be increased without sacrificing flexibility by increasing the thickness (or rigidity) of the supports 116 but keeping the flexure 114 relatively small or flexible.

The flexure assembly 110 can be configured to provide any desired degree of flexibility to the implant 100. As discussed above, some embodiments of the implant are configured to provide a certain degree of flexibility along a certain axis dependent on the desired target location of the implant within the spine. Accordingly, the flexure assembly can have any suitable flexibility that allows it to impart the desired flexibility to the implant (either alone, or in combination with other flexure assemblies). For example, some embodiments of the flexure assembly are configured to provide between 0 degrees and 20 degrees of flexing (or any subrange thereof) along any particular axis. For example, some embodiments are configured to provide 2±2, 3±2, 4±3, 5±3, 6±2, 6±3, 8±3, 7±5, 10±4 , at least 1, at least 2, at least 3, at least 4, at least 5, at least 6, at least 7, at least 8, or at least 9 degrees, or any other suitable degree of flexing along each or any of the flexion/extension axis, the lateral bending axis, or the axial rotation axis.

To illustrate how the flexibility of the flexure assembly 110 can contribute to the flexibility of the implant as a whole, if a flexibility of 8 degrees±3 degrees along the flexion/extension axis is desired, and if the implant has only one flexure assembly, then such flexure assembly can have a flexibility of 8 degrees±3 degrees along the flexion/extension axis. If the implant has two or more flexure assemblies, then the flexure assemblies can be more flexible along the flexion/extension axis of the implant such that the cumulative flexibility of the implant along the flexion/extension axis is 8 degrees±3 degrees.

Similarly, the flexure assembly 110 can have any suitable stiffness as needed to contribute to the overall stiffness of the implant 100 (as discussed above). For example, where a single flexure assembly is used, the flexure assembly can have a stiffness of up to 100% of the stiffness of the implant (or of a portion of the implant that is disposed between two endplates 102), whereas some embodiments of a flexure assembly contribute to only a portion of the stiffness of the implant (e.g., where additional flexure assemblies, compression-based elements, or other components contribute to the stiffness of the implant). That said, in some embodiments, the flexure assembly (or any of its constituent components, such as the flexure 114, as discussed in more detail below) is configured to withstand at least 10% of the compressive force to which the implant will be subject (e.g., at least 25%, at least 50%, at least 75%, or even up to 100% of the compressive force). Thus, although the implant can have any number of flexure assemblies, a single flexure assembly (or a relatively small number, such as between 1 and 5, or fewer than 10) is sufficient, in some embodiments, to withstand the forces to which the implant will be subjected.

In line with the foregoing, the flexure assembly 110 can have any suitable configuration allowing it to impart the desired flexibility and stability to the implant 100. In this regard, and as illustrated by FIG. 5, a flexure assembly 110 can generally be referred to as having a height h, a width w, and a length l. It is important to note that these designations are for reference only, and do not limit the flexure assembly to any particular orientation with respect to other portions of an implant 100. For example, although some embodiments of a flexure assembly are configured such that the height h of the flexure assembly bridges the gap between the implant's endplates 102 (e.g., generally extending in a rostral-caudal direction), some embodiments are arranged in alternative configurations (e.g., such that the width w extends in a rostral-caudal direction, the length l extends in a rostral-caudal direction, a portion of the flexure assembly along a diagonal extends in a rostral-caudal direction, or the flexure assembly is disposed in an a different orientation with respect to one or more of the endplates). Notwithstanding the foregoing, for purposes of simplicity, the height h of a flexure assembly is generally referred to as the direction aligned substantially parallel to a central axis of the flexure, with the length l and the width w running substantially perpendicular to the central axis of the flexure. Additionally, although the width is generally described as the direction bordered by a first support 116a and a second support 116b (as discussed below), the length and the width are reversed in some embodiments. Accordingly, for the purposes of this disclosure, a description of a width with characteristic A and a length with characteristic B also includes a description of a width with characteristic B and a length with characteristic A.

The flexure assembly 110 can have any suitable dimensions allowing it to perform its intended functions. In some embodiments, the flexure assembly provides a minimum height of 5 mm for cervical uses and 7 mm for lumbar uses. Moreover, it should be noted that, in some cases, mechanisms that provide stable motion but that cannot fit within a corresponding height envelope can pose the risk of overstuffing the space and damaging surrounding soft tissues and facet joints. Thus, some embodiments of the flexure assembly have a height h that equals a distance between the first endplate 102a and the second endplate 102b (±5%). That said, in some embodiments, the height of the flexure assembly is less. For example, some embodiments of the implant include two or more flexure assemblies (or other components) stacked on top of one another, such that the heights of each flexure assembly (or other components) summed together would equal the distance between the endplates (±5%). In some embodiments, the height of the flexure assembly is between 1 mm and 100 mm (or any subrange thereof). For example, in some embodiments, the height is between: 4 mm and 10 mm, 5 mm and 12 mm, 7 mm and 17 mm, or any other subrange between 1 and 40 mm.

Similarly, the flexure assembly 110 can have any suitable width that allows it to function as intended. In this regard, some embodiments of the flexure assembly fit within the circumference of a vertebral body. Additionally, it should be noted that cervical bodies are generally smaller than lumbar bodies. In any case, fitting within the size constraints of the index level is, in some cases, preferred. In some embodiments, the width is less than a width of the endplates (such as less than 100% of the endplate width, or less than 90%, 80%, 70%, 75%, 60%, 50%, 40%, 25%, 10%, or any other percentage of the endplate width). In some embodiments, the width of the flexure assembly is between 1 mm and 100 mm (or any subrange thereof, such as the subranges shown above with respect to height).

Similarly, some embodiments of the flexure assembly 110 have a length that is less than a length of the endplates (such as less than 100% of the endplate length, or less than 90%, 75%, 50%, 40%, 25%, 10%, or any other percentage of the endplate length). In some embodiments, the length of the flexure assembly is between 1 mm and 100 mm (or any subrange thereof, such as the subranges shown above with respect to height).

In addition to the flexure assembly 110 having any suitable dimensions, the various components of the flexure assembly, such as the loading regions 112, the supports 116, and the flexure 114 can have any suitable dimensions, as discussed in more detail in the descriptions of the individual components below.

The flexure assembly 110 can have any suitable shape that allows it to function as described herein, including by having one or more tension-based flexures 114. Indeed, in some embodiments, the flexure assembly (or a portion thereof): has an N-shape, resembles a portion of the Greek fret or Greek key, resembles the shape-of an upside-down U (or first squared bracket) coupled together with a right-side-up U (or second squared bracket), is wave-shaped, comprises an undulated portion, is sinusoidal, resembles a square wave form, resembles a rectangular wave form, resembles a sawtooth waveform, resembles a triangle waveform, or has any other suitable shape that allows the flexure assembly to function as described herein. By way of non-limiting illustration, FIG. 4 shows an embodiment in which the flexure assembly 110 resembles a section of the Greek key; FIG. 11 shows an embodiment in which the flexure assembly 110 resembles a right-side-up U coupled to an upside down U with the tension-based flexure 114 comprising part of and coupling together the two U-shaped portions; and FIG. 13 shows an embodiment in which the flexure assembly 110 appears to comprise a first squared bracket in a first orientation, coupled to a second squared bracket in a second, opposite (or substantially opposite) orientation. Thus, in some cases, the flexure assembly comprises one or more first supports 116a (e.g., vertical, diagonal, or any other suitable supports) that are coupled to one or more tension-based flexures 144 (e.g., via one or more arched portions, transverse portions, diagonal portions, or any other suitable couplers), which in turn are coupled to one or more second supports 116b (e.g., via one or more arched portions, transverse portions, diagonal portions, or any other suitable couplers).

The flexure assembly 110 can include any component that allows it to support a load (e.g., a load placed on the endplates 102 of the implant 100) using tension. For example, (as mentioned above) some embodiments of a flexure assembly include one or more loading regions 112, one or more flexures 114, and one or more supports 116.

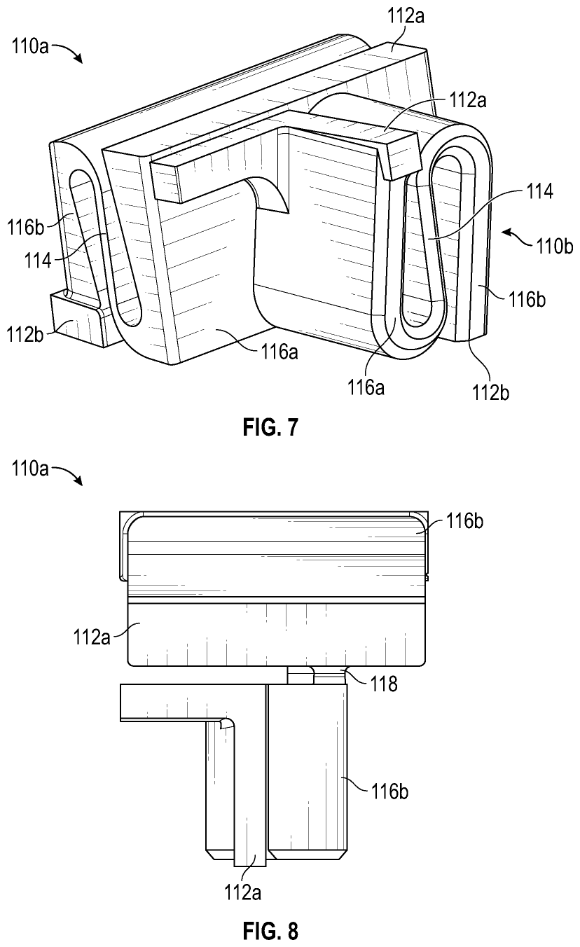

For embodiments of a flexure assembly 110 that include one or more loading regions 112, the loading regions can include any suitable component configured to receive a load, to couple with an endplate 102, or to comprise an endplate. In some cases, as it can be desirable to avoid contact stress, a flexure assembly that avoids contact loading between components is provided. For example, some embodiments of the loading region include one or more plates, flanges, bars, bases, scaffolds, porous structures, contact surfaces, couplers, or even just a surface (of the flexure assembly or of any of its constituent components) configured to transfer a load from an endplate 102 (or from a bone) to the flexure assembly. The loading region can be any shape, including circular, semi-circular, triangular, square, rectangular, trapezoidal, pentagonal, hexagonal, star-shaped, T-shaped, L-shaped, polygonal, or any other regular or irregular shape. By way of non-limiting illustration, FIG. 4 shows an embodiment of the flexure assembly 110 with a first loading region 112a and a second loading region 112b, each comprising a rectangular bar or elongated member configured to receive a load. By way of further illustration, FIG. 5 shows an embodiment of the flexure assembly 110 with a first loading region 112a and a second loading region 112b, each comprising a square, rectangular, or polygonal plate configured to receive a load. To further illustrate, FIG. 7 shows an embodiment of the first flexure assembly 110a with: (a) a first loading region 112a that simply comprises an upper surface of a first support 116a (having a generally rectangular or flat surface); and (b) a second loading region 112b that includes a base having a larger surface area than a lower surface of a second support 116b. FIG. 7 also shows an embodiment of the second flexure assembly 110b with: (a) a first loading region 112a that includes an L-shaped component coupled to the first column or support 116a of the first flexure assembly 110a and to the first column or support 116a of the second flexure assembly 110b; and (b) a second loading region 112b, which includes a bottom surface of a second support 116b.

It is important to note that some embodiments of the flexure assembly 110 optionally do not have a separate plate, flange, bar, or other component to act as a loading region 112. For example, in some embodiments, the supports 116 of the flexure assemblies couple directly to the endplates 102 of the implant 100 (such that the endplates themselves act as the loading regions). In some embodiments, the supports take the load directly, such that a separate portion designated as a loading region is not necessary (instead, the ends or end portions of the supports taking the load act as the loading regions).

In some embodiments, one or more loading regions 122 of the flexure assembly 110 are configured to perform additional functions, such as to: couple other components together, define one or more passages in the implant 100 lend additional structural support (e.g., to the endplates 102), or otherwise improve the functionality of the implant. In accordance with some embodiments, a passage from the vertebral endplate into the disc space allows for nutrient flow to the healing mass of new tissue. On this point, it is noted that the vertebrae are generally the source of nutrient supply to a disc space. Thus, in some embodiments, a flexure assembly that allows for the nutrients to flow freely from the vertebral body into the disc space is provided (e.g. a graft window). By way of non-limiting illustration, FIG. 9 shows an embodiment of the first loading region 112a of a second flexure assembly 110b that performs some of the aforementioned functions, as it has an L-shape that allows it to define a window 126 in the implant 100, and it connects to a first endplate 102a (thereby adding structural support).

The loading region 112 can have any suitable dimensions allowing it to bear a load. For example, some embodiments of the loading region have a length or width approximately equal to the length or width of the flexure assembly (e.g., 90%±10% of a length or width of the assembly), but in some embodiments the length or width is substantially less (e.g., as low as 1% of the length or width of the flexure assembly). In many embodiments, the loading region has a height that is substantially less than the height of the flexure assembly (e.g., as shown by the loadings regions 112a and 112b of FIG. 5). For instance, in some embodiments, the height of the loading regions is between 1% and 20% of the height of the flexure assembly, or any subrange thereof (e.g., 10%±8%). In some embodiments, the loading region is just a surface of a portion of the flexure assembly, and is therefore 2-dimensional and lacks a height altogether.

In some embodiments with more than one loading region 112, the loading regions are aligned (e.g., such that the first loading region 112a would partially or completely occlude or overlap with the second loading region 112b when viewed from directly above), but in some embodiments, the loading regions are offset. For example, the loading regions 112a and 112b of the flexure assembly 110 in FIG. 4 are substantially aligned, but the loading regions 112a and 112b of the implant 100 in FIG. 7 are offset to such a degree that they do not overlap.

Turning now to the flexure 114 of the flexure assembly 110, the flexure can include any component with sufficient tensile strength to bear the load placed on it from use of the implant 100 and sufficient flexibility to provide the desired range of motion along each axis. In some embodiments, the flexure is configured to eliminate stress concentrations and plastic deformation along its length. In some cases, this ensures that the flexure bends at stress levels that are well below its yield strength. Selecting geometries that are tailored to the material, the desired performance stiffness, and the desired range of motion is, in accordance with some embodiments, enabled by this flexure. For example, the flexure can include a thin sheet of material (e.g., metal, plastic, carbon fiber, polymer material, fabric, ceramic, natural material, synthetic material, or any other material), or it can include one or more strands, cords, sheets, walls, rods, hooks, chains, bars, filaments, or any other tension-bearing components that can bend well below their yield stress. By way of non-limiting illustration, FIGS. 7, 11, and 12 show flexures 114 that include relatively straight and relatively narrow sheets of material supported by supports 116a and 116b on either end.

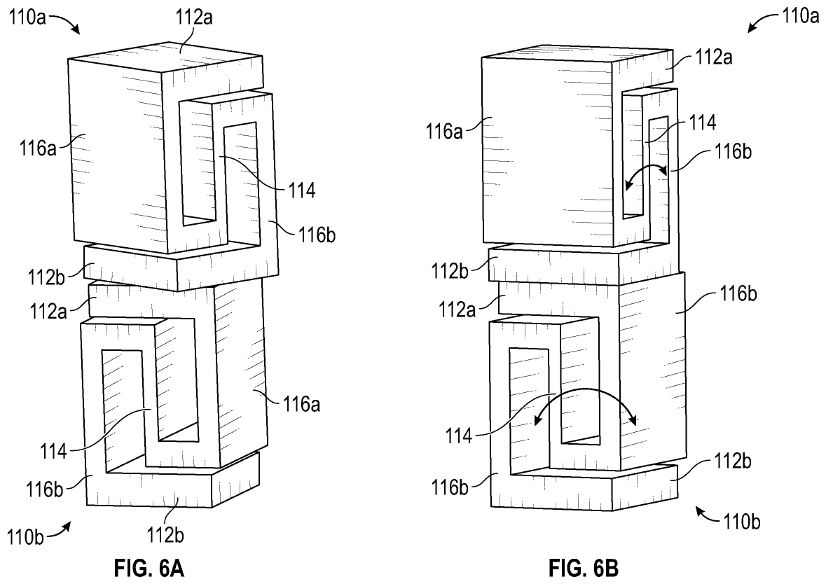

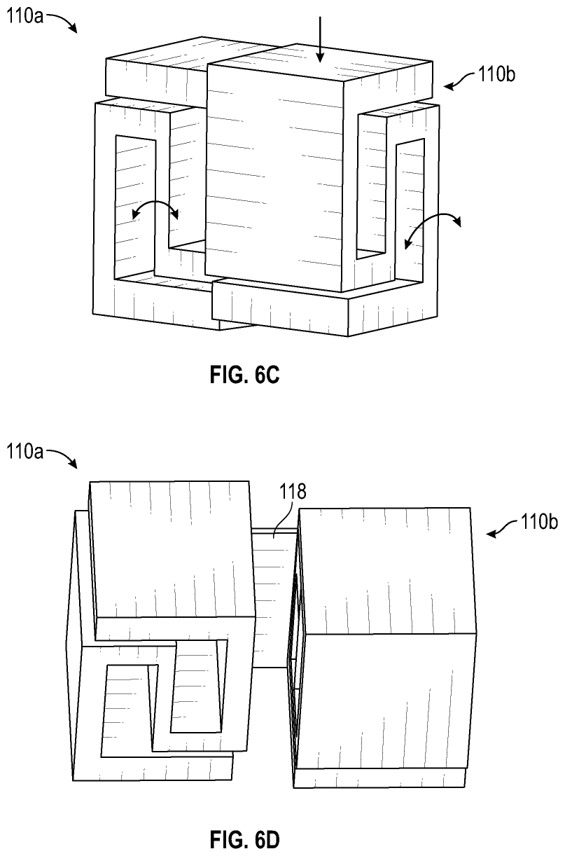

In some embodiments, the flexibility of the flexure assembly 110 can be adjusted by adjusting the flexure 114, including by adjusting the material the flexure comprises, by adjusting the shape of the flexure, by adjusting the dimensions of the flexure, by perforating the flexure, or in any other suitable manner. For example, a flexure 114 with a narrow length and width (e.g., a large height to length or height to width aspect ratio, as shown in FIG. 5) can be flexible along each axis, but that flexibility can be decreased along one or more axes by increasing the length or width of the flexure 114 (as seen in FIGS. 6A-6D in comparison to FIG. 5). In some embodiments, the flexibility of the flexure is tailored to achieve the desired flexibility of the flexure assembly (as discussed above) to consequently achieve the desired flexibility of the implant 100 as a whole.

Although the flexure 114 can be positioned anywhere within the flexure assembly 110, some embodiments include at least a portion of the flexure in the center of mass of the flexure assembly for added stability. In some cases, the flexure is positioned such that all portions of the flexure receive an approximately equal share of the total load. In this regard, in accordance with some embodiments, placing the center of rotation of the device at the spinal level's center of rotation helps provide for anatomical motion. Furthermore, in some embodiments, the flexure assembly provides for a moving center of rotation that approximates the helical axis of motion of the spine level.

The flexure 114 can have any dimensions that allow it to have sufficient tensile strength to support the load placed on the implant 100 and to have the desired flexibility along each axis. Generally speaking, the aspect ratio of some embodiments of the flexure can be much different than the aspect ratio required for a compression-based stem. For example, the flexure can have a much greater height-to-width or height-to-length ratio (or both) without danger of buckling or breaking.

According to some embodiments, the flexure 114 has a height between 1% and 100% of the height of the flexure assembly 110 (or any subrange thereof, such as 50%±10%, 60%±10%, 70%±10%, 80%±10%, or 90%±10%). That said, some embodiments of the flexure are shorter than the flexure assembly (e.g., the supports extend farther than the flexure on either side of the flexure's height) to ensure that the flexure is placed in tension (and not compression) when the implant 100 is loaded. In some embodiments, at least one of the width and the length of the flexure is substantially less than the height of the flexure, such as less than 50% of the height (or any other number less than 50%, such as less than 40%, 30%, 25%, 20%, 15%, 10%, 5%, or even 1% of the height). That said, the length of the flexure can be any length up to 100% of the length of the flexure assembly, and the width can be any width up to 100% of the width of the flexure assembly (although this would require an alternative configuration of the supports in some cases, which in some embodiments are situated on either side of the flexure, thereby adding to the width of the flexure assembly). In short, the flexure can be customized by modifying the height-to-length-to-width aspect ratio as needed to form a flexure with the desired flexibility along the proper axes. Thus, the aspect ratio can be any ratio of h-l-w (where h is the height, l is the length, and w is the width) with h being any number between 1 and 100 (or any subrange thereof), l being any number between 1 and 100 (or any subrange thereof), and w being any number between 1 and 100 (or any subrange thereof), but with h being at least two times at least one of l and w. As examples: 10-1-1, 10-5-2, 10-10-1, 15-2-1, 8-9-1, and 12-1-8 are all possible h-l-w aspect ratios that may be useful for a flexure. In some embodiments, h is at least three times at least one of l and w, at least four times at least one of l and w, at least five times at least one of l and w, or otherwise substantially larger than at least one of l and w. In some embodiments, the aspect ratio of the flexure is configured to ensure that the flexure assembly has a desired flexibility along one or more axes, as described above.

The flexure 114 can also be any shape suitable for being placed under tension. For example, some embodiments of the flexure include one or more substantially linear, planar, cylindrical, columnar (e.g., cylindrical-columnar, square-columnar, triangular-columnar, polygonal-columnar, or otherwise configured in a column), tubular, sheet-like, undulated, coiled, wavy, or any other suitable shaped components. By way of non-limiting illustration: FIG. 5 shows an embodiment of a substantially linear rectangular-columnar flexure 114; FIG. 6A shows an embodiment of a substantially planar rectangular-columnar flexure 114; and FIG. 7 shows an embodiment of a substantially planar rectangular-columnar flexure.

The flexure 114 can also have any suitable orientation. For example, in some cases, a long or longitudinal axis of the flexure substantially aligns (e.g., is substantially coaxial, or is within 1 degree, 2 degrees, 3 degrees, or 5 degrees of coaxial) with a long (or longitudinal) axis of the flexure assembly 110, or with an axis of compression of the implant 100. Indeed, in some cases, when the long axis of the flexure is coaxial with the implant's axis of compression, this places minimal stress on the flexure. That said, in some cases, one or more additional characteristics (e.g., resistance along one or more axes) can be achieved by orienting the flexure differently with respect to the axis of compression. In this regard, some embodiments include a flexure with a long axis that is not coaxial or substantially coaxial with the implant's axis of compression, such as by being off by between 1 degree and 25 degrees or any suitable subrange thereof (e.g., between 1-5 degrees, 3-10 degrees, 5 -15 degrees, or any other suitable subrange). In some embodiments, multiple flexures are included, with each flexure having a long axis that is not coaxial (or substantially coaxial) with the implant's axis of compression, but the sum of the tension on all such flexures is coaxial (or substantially coaxial) with the implant's axis of compression.

Related to the foregoing, and generally speaking, the implant's axis of compression includes any axis along which at least a portion of the first endplate 102a (and in some cases, the first endplate as a whole) moves toward at least a portion of the second endplate 102b (and in some cases, the second endplate as a whole). In this regard, in some cases, the endplates move directly toward each other, translationally moving along the axis of compression. That said, in some embodiments, the endplates pivot such that only a portion of each endplate moves toward the other endplate (e.g., a compressive force on a posterior portion of the endplates can cause the posterior portions of the endplates to move toward each other along the axis of compression). Additionally, it should be noted that while some embodiments of the endplates are substantially parallel to each other, in some embodiments, the endplates are set at a different angle with respect to each other. Indeed, in some cases, the endplates are between 0 and 25 degrees of parallel to each other, or any suitable subrange thereof (e.g., between 0 and 15 degrees, between 5 and 10 degrees, between 2 and 20 degrees, or any other suitable subrange). In some cases, such as where the endplates are angled, the axis of compression is not necessarily perpendicular to the endplates. Thus, the axis of compression may generally extend between a portion of the first endplate to a corresponding portion of the second endplate (e.g., between the anterior-most point at the midline of both endplates). Stated another way, if one endplate is angled upward 5 degrees from horizontal and the other endplate is angled downward 5 degrees from horizontal, the axis of compression may be generally perpendicular to the first endplate, the second endplate, or to an average angle of both endplates (in this case, horizontal, as the positive and negative 5-degree tilts of the respective endplates cancel each other out when averaged together).

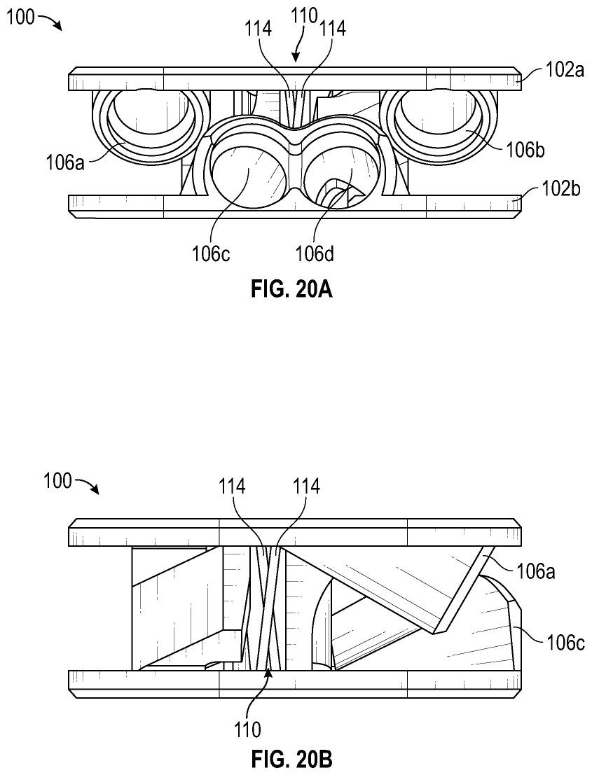

By way of non-limiting illustration, FIGS. 20A-20B show some embodiments of implants 100 with two flexures 114 that are at an angle with respect to the implant's 100 axis of compression, where the flexures 114 cross each other such that when the flexures 114 are both in tension, the sum of the tension of both flexures 114 together is approximately coaxial with the implant's 100 axis of compression. That said, FIG. 12 shows some embodiments of an implant 100 with a flexure 114 that is oriented substantially coaxial with the implant's 100 axis of compression (e.g., substantially perpendicular to the endplates 102a, 102b (or to an average angle of the endplates), or substantially parallel to the load placed on the implant).

It should be noted that a normal human disc typically resists compression, is relatively flexible in flexion/extension, is less flexible in lateral bending, and is even less flexible in axial rotation. Also, a human disc largely resists relative translation between the vertebral endplates. In this regard, getting each relative stiffness right at the same time, without sliding contact, and with a proper center of rotation is a feature of some embodiments of the described implant 100—a feature that is not found in most, if any, competing devices. Moreover, providing all that with a graft window for nutrient flow is also a feature of some embodiments of the described implant—again something that is not found in most, if any, competing devices.

Where the flexure assembly 110 includes one or more supports 116, the supports can include any component allowing load to be transferred from the loading regions 112 to the flexure 114 in such a manner as to place the flexure under tension. Some embodiments include at least two supports, such that the first support 116a can transfer the load from a first loading region 112a to a first end 114a of the flexure disposed opposite the first loading region, and the second support 116b can transfer the load from a second loading region 112b to a second end of the flexure 114b disposed opposite the second loading region (as shown in FIG. 4). This contrasts with a compression-based stem (as shown in FIG. 3) in which the load from a first loading region 62a is transferred directly to a first end 64a of the stem and the load from a second loading region 62b is transferred directly to a second end 64b of the stem, such that the stem is placed under compression.

The supports 116 can include any suitable components that provide the desired support, including one or more pillars, columns, rods, walls, sheets, beams, posts, bars, or other supports. The supports can have any supportive shape, such as cylindrical, conical, square-beam-shaped, V-shaped, triangular, sheetlike, or otherwise shaped. By way of non-limiting illustration, FIG. 4 shows an embodiment comprising pillar-like supports 116a and 116b. FIGS. 5-6D show embodiments comprising sheet-like supports 116a and 116b. FIG. 7 shows a first flexure assembly 110a with contoured supports (e.g., thicker near loading regions 112a and 112b, and thinner near the flexure 114, with curved contours joining the supports to the flexure). FIG. 12 shows an embodiment having a first support 116a and a second support 116b, each having a sloped surface to provide additional support near the loading regions 112a and 112b. FIG. 13 shows an embodiment comprising multiple flexure assemblies 110a, 110b, 110c, and 110d with supports 116a and 116b that have a general rectangular block configuration. FIG. 14 shows an embodiment comprising a second flexure assembly 110b with slanted supports 116a and 116b.

The supports 116 can also have any suitable dimensions allowing them to support a tension-based flexure 114. For example, the supports can have any suitable length, but some embodiments of the supports have a length approximately equal to the length of the flexure assembly (e.g., 90%±10% of the flexure assembly length). Some embodiments have a length of approximately between 1% and 15% of a length of the flexure assembly. The supports can also have any suitable width, but in some embodiments the width is sufficient to ensure that the supports are substantially rigid (e.g., having a flexibility of less than 10% of the overall flexibility of the flexure assembly 110, a flexibility of less than 1 degree, or otherwise having a rigidity sufficient to prevent buckling and breaking of the flexure assembly when placed under a load as required by the implant 100). Some embodiments of the supports have a width between 1 mm and 10 mm, or any subrange thereof. Some embodiments of the supports have a width between 1% and 10% of the width of the flexure assembly, or any subrange thereof. The supports can also have any suitable height that allows them to transfer a compressive force received at one end (the end by the loading region) to a tensile force at the other end (through its coupling with the flexure 114. For example, in some embodiments the supports have a height that is greater than a height of the flexure. In some cases, the supports have a height between 1% and 20% greater than the height of the flexure (or any subrange thereof, such as 5%±3%), and in some cases, the supports have a height between 0.1 mm and 20 mm greater than the height of the flexure. In some embodiments, the supports have a height of between 50% and 100% of the height of the flexure assembly (or any subrange thereof, such as 85%±10%).

In some embodiments, the supports 116 are offset from one another in one or more directions. For example, some embodiments of the supports are offset (e.g., by up to 50% or any percentage below that, such as 25%, 10%, 5%, 1%, or another percentage) along the axis of the length of the flexure assembly 110 or the axis of the height of the flexure assembly (or both). By way of non-limiting illustration, FIG. 5 shows an embodiment in which the supports 116a and 116b are offset along the length l and height h of the flexure assembly 110. In many cases, the supports are naturally spaced apart along the width w (e.g., offset by between 100% and 1,000%, or any subrange thereof), as this allows one support to be positioned on either side of the flexure 114.

The flexure 114 and supports 116 can couple together in any suitable manner. For example, in some embodiments, the flexure and support are joined by one or more intermediate components 115. In some embodiments, at least one of the flexure and the supports is contoured such that they join together directly. Whether the supports include an intermediate component or portion or are contoured to join to the flexure directly, they can include any suitable feature, such as one or more arches, squared couplers, horizontal couplers, diagonal couplers, semi-circles, bends, curves, processes, protrusions, overhangs, ledges, outcroppings, or any other suitable contours or components allowing them to join to the flexure. By way of non-limiting illustration, FIG. 5 shows an embodiment of the flexure 114 joined to supports 116a and 116b via one or more intermediate components 115a and 115b. In contrast, FIG. 7 shows flexure assemblies 110a and 110b having contoured supports 116a and 116b that couple directly to flexures 114.

Although some embodiments of the flexure assembly 110 include one flexure 114 and two supports 116 (e.g., one support for one end of the flexure and one support for the other end), other arrangements with different numbers of flexures or supports are also possible. For example, in some cases, a single support (e.g., a beam or another support) supports two or more flexures. In some cases, a single side of a flexure is supported by two or more supports. Indeed, in some cases, the flexure assembly is segmented so as to facilitate the presence of one or more windows 126 (as discussed in more detail below). Accordingly, in some cases, the supports can include one or more arches or other types of supports configured to define one or more negative spaces while still providing the compression-based support necessary for placing the flexure in tension.

Although the loading regions 112, flexure 114, and supports 116 can be separate components, some embodiments of the flexure assembly 110 are integrally formed as a single piece. For example, in some cases, at least the flexure and the supports are integrally formed as a single piece. Where one or more components of the flexure assembly are provided as a separate piece, the components can be joined together in any suitable manner, such as via one or more nails, screws, bolts, staples, eyelets, magnets, adhesives, welds, interference fits, friction fits, mechanical engagements, tongue-and-groove connections, snaps, ties, rivets, pins, fasteners, stakes, wire ties, or any other coupling mechanisms.

Although some embodiments of the implant 100 include only a single flexure assembly 110, some embodiments include multiple flexure assemblies, such as a first flexure assembly 110a, a second flexure assembly 110b, a third flexure assembly 110c, a fourth flexure assembly 110d, or any other number of additional flexure assemblies. In this regard, some embodiments of the flexure assembly are more flexible (or less flexible) along one or more particular axes than are other embodiments. Accordingly, coupling such embodiments together can result in a device with the desired flexibility and the desired strength along any or all axes. Multiple flexure assemblies can also operate independently of one another, thereby adding flexibility and stability capabilities to multiple portions of the implant.

Where multiple flexure assemblies 110 are used together, they can be coupled or otherwise arranged in any suitable manner, such as by being stacked (e.g., arranged in series), placed end to end, placed side-by-side (e.g., arranged in parallel), being offset from each other, or placed in any other suitable configuration. By way of non-limiting illustration, FIGS. 6A and 6B show some embodiments depicting flexure assemblies 110 in a series arrangement, whereas FIGS. 6C and 6D show some embodiments having a parallel arrangement. In some embodiments, multiple flexure assemblies may be arranged side-by-side, but coupled together in such a manner so as to cause the load path to transmit at least a portion of the load (and in some cases, the whole load) from one flexure to another flexure in series. Thus, multiple flexure assemblies can operate entirely in series, entirely in parallel, or partially in series and partially in parallel. By way of non-limiting illustration, FIG. 12 shows an embodiment of the implant 100 with a first flexure assembly 110a coupled to a second flexure assembly 110b such that a portion of the load path is transferred from one flexure assembly to the other in series, while the flexure assemblies still each take some of the load on their own, thereby operating partly in parallel.

Where multiple flexure assemblies 110 are used, the flexure assemblies can be placed at any angle or orientation with respect to each other. For example, some embodiments of the flexure assembles have the same orientation along every axis, but some embodiments have at least one flexure assembly that is rotated with respect to at least one other flexure assembly along at least one axis in any amount between 0 degrees and 360 degrees, or any subrange thereof. As an example, one flexure assembly is rotated between 15 degrees and 95 degrees, between 35 degrees and 55 degrees, or any other suitable range of rotation, along the torsion axis with respect to another flexure assembly. As another example, one flexure assembly can be rotated 45 degrees (±10 degrees) along the flexion/extension axis as well as 120 degrees (±10 degrees) along the lateral bending axis. As another example, one flexure assembly can be rotated 180 degrees (±10 degrees) of another flexure assembly along one or more axes, and a third flexure assembly can be rotated in any amount along any axis with respect to either or both of the other two flexure assemblies. By way of non-limiting illustration, FIG. 6A shows an embodiment of the first flexure assembly 110a rotated between 10 degrees and 40 degrees along a torsion axis with respect to a second flexure assembly 110b. FIGS. 6B-6D show a first flexure assembly 110a rotated approximately 45 degrees (±5 degrees) along a torsion axis with respect to a second flexure assembly 110b.

Furthermore, in some embodiments, multiple flexure assemblies 110 are aligned with each other, and in some embodiments, multiple flexure assemblies are offset from one another (in any suitable amount, such as anywhere between 0% and 500%, or any subrange thereof). For example, in some embodiments, a first flexure assembly is positioned next to and aligned with a second flexure assembly such that a face of the first flexure assembly is flush with a face of the second flexure assembly. As another example, in some embodiments with multiple flexure assemblies, the flexure assemblies are slightly offset (e.g., between 1% and 5% offset, between 2% and 10% offset, between 5% and 20% offset, or otherwise offset) from each other such that one flexure assembly is higher, farther forward, farther backward, or to farther to a side than the other one. In some embodiments, the flexure assemblies are greatly offset such that they do not align at all.

In some embodiments, the rotation, position, or other configuration of one or more flexure assemblies 110 is selectively adjustable or otherwise customizable. In this regard, the flexure assembly can be adjustable or customizable in any suitable manner, including by being specifically designed for a patient based on the patient's particular needs (e.g., as determined by X-ray, imaging, or in any other suitable manner), having the endplates 102 be configured to couple with the flexure assemblies in one of multiple orientations, through the use of one more adjustment mechanisms, or in any other suitable manner.

In some embodiments, multiple flexure assemblies 110 are coupled together. Where multiple flexure assemblies are coupled together, they can be coupled in any suitable manner, such as through welding, being formed as an integral part, adhering together using one or more adhesives, via one or more couplers 118, via one or more fasteners, via one or more frictional or mechanical engagements, or through any other suitable manner. Where a coupler is used, the coupler can include any suitable selective, permanent, or semi-permanent coupling component, such as one or more one or more nails, pins, screws, bolts, rods, sheets, staples, eyelets, magnets, hook-and-loop fasteners, adhesives, welds, interference fits, friction fits, tongue-and-groove connections, snaps, ties, rivets, wire ties, or any other type of coupler. In some cases, the coupler itself includes one or more flexure assemblies or parts thereof (e.g., having any of the properties relating to flexure assemblies as discussed herein), thereby allowing the coupler to have enhanced stability and flexibility. The coupler can also have any suitable dimensions (e.g., any width, height, and length suitable for coupling multiple flexure assemblies together). By way of non-limiting illustration, FIG. 6D shows an embodiment of a first flexure assembly 110a and a second flexure assembly 110b coupled together via a coupler 118 that includes a flexible piece of material (or any other suitable material) coupled to both flexure assemblies. FIGS. 8-10 show some embodiments of implants 100 with multiple flexure assemblies 110a and 110b coupled together via one or more couplers 118, where such coupler or couplers includes a thin, flexible piece of material (or any other suitable material) joining a first support 116a of a first flexure assembly 110a to a second support 116b of a second flexure assembly 110b such that the respective flexure assemblies are perpendicular with respect to each other. FIG. 12 shows a pair of flexure assemblies 110a and 110b that are integrally joined together, such that a coupler 118 is substantially in the form of an extension of the flexure assemblies allowing them to merge together into a unified construct. FIG. 13 shows an X-shaped coupler 118 configured to join four flexure assemblies 110a, 110b, 110c, and 110d together.

In some embodiments with multiple flexure assemblies 110, a first flexure assembly 110a can primarily govern flexibility along the lateral bending axis, and a second flexure assembly 110b can primarily govern flexibility along the flexion/extension axis (as shown in FIGS. 11-12), or vice versa. In some embodiments with multiple flexure assemblies, all such flexure assemblies are configured to sum together to provide the desired flexibility along any particular axis (e.g., the flexion/extension axis, the lateral bending axis, or the torsion axis).

Where multiple flexure assemblies 110 are used, the flexure assemblies can have any size with respect to each other. For example, in some embodiments, a first flexure assembly 110a has a height between 50% and 200% of a height of a second flexure assembly 110b, or any subrange thereof. For example, in some cases, the first flexure assembly's height is 95%±4% the height of the second flexure assembly (or 92%±5%, 105%±5%, 110%±5%, 65%±15%, or any other suitable height differential). By way of non-limiting illustration, FIG. 12 shows an embodiment of a first flexure assembly 110a that is slightly shorter than a second flexure assembly 110b, such that a first endplate 102a and a second endplate 102b are closer together near the first flexure assembly than near the second flexure assembly. Similarly, either or both of the width or the length of one flexure assembly can be anywhere between 20% and 1000% (or any subrange thereof) of the width or length of another flexure assembly. For example, in some embodiments, a first flexure assembly 110a has a length (or width) that is 200%±50% (or 110%±5%, 150%±15%, 250%±50%, or any other suitable percentage range) the length (or width) of a second flexure assembly. By way of non-limiting illustration, FIGS. 7 and 8 show embodiments of a first flexure assembly 110a that has a length that is greater than a length of a second flexure assembly 110b, wherein the first and second flexure assemblies have a substantially similar width.

Some embodiments include one or more additional elements to modulate any of the properties of the described implant 100. For example, some embodiments include one or more additional elements for increasing the relative stiffness of the implant, such as one or more reinforcement elements 122. Reinforcement elements can include one or more springs 128, coils, coil packs, beams, bars rods, struts, posts, torsional elements, frame, lattices, resilient members, scaffolds, porous structures, trusses, supports, fills, woven supports, twisted supports, interconnecting supports, matrices, compressive elements, or any other additional elements. In some embodiments, an array 120 of reinforcement elements is provided.

In some embodiments, the array 120 of reinforcement elements 122 forms a porous structure. The porous structure can include any structure defining gaps, spaces, air pockets, voids, cavities, hollows, or other pores capable of promoting bone growth (e.g., cortical bone growth, trabecular bone growth, or other bone growth).

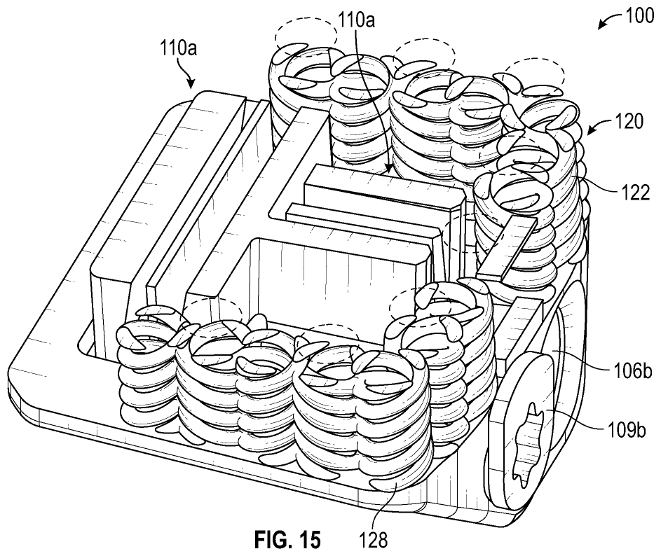

The reinforcement elements 122 can include any component capable of increasing the relative stiffness, strength, compliance, or integrity of the implant 100, of exerting a compressive load, of encouraging bone growth, or of modulating any of the other properties of the implant (e.g., flexibility along any particular axis). For example, the reinforcement elements can include one or more supports, meshes, grids, strands, filaments, foams, lattices, matrices, sponge-shaped supports, supports that resemble cancellous or trabecular bone, wires, grates, springs, coils, frames, frets, screens, reticulations, or other auxiliary components. By way of non-limiting illustration, FIGS. 14-15 show some embodiments of the implant 100 having an array 120 formed of a plurality of compression-based reinforcement elements 122.

In some embodiments, the reinforcement elements 122 include at least a first reinforcement element 122a and a second reinforcement element 122b (and any number of additional reinforcement elements). Where the implant 100 includes multiple reinforcement elements 122 (e.g., an array 120 of such elements), the reinforcement elements can be arranged in any manner suitable for achieving their desired functions. For example, in some embodiments, reinforcement elements are disposed in one or more portions of the implant not occupied by flexure assemblies 110. In some embodiments, reinforcement elements are disposed around all or part of a perimeter of the implant (such as along one or more of a dorsal face, a ventral face, a left face, or a right face. In some embodiments, one or more reinforcement elements are disposed within an interior of the implant (e.g., adjacent to, surrounding, filling, flanking, or between one or more flexure assemblies). In some embodiments with multiple reinforcement elements, the reinforcement elements are interlaced, interwoven, interlocking, coupled together, included in a repeating pattern, or included in a mirrored pattern (which in some cases makes the reinforcement elements easier to manufacture or adds additional strength), but the reinforcement elements may also be included in any other pattern or arrangement. By way of non-limiting illustration, FIG. 14 shows an embodiment of the implant 100 with an array 120 of reinforcement elements 122, wherein the reinforcement elements 122 include coils, and wherein a first reinforcement element 122a includes a right-hand (clockwise) coil and a second reinforcement element 122b includes a left-hand (counterclockwise) coil, thus lending the array a repeating mirrored pattern around a portion of a perimeter of the implant. In some embodiments, one or more coils are separate and discrete from one or more adjacent coils. In some embodiments, however, one or more coils are coupled to, integrally formed with, tangentially coupled to, or otherwise connected with one or more adjacent coils.

In some embodiments, the reinforcement elements 122 can couple to any suitable portion of the implant. Indeed, in some embodiments, the reinforcement elements are coupled to the endplates 102 (or to any other suitable component, such as one or more flanges 104 or flexure assemblies 110) of the implant 100. While this coupling can be done in any suitable manner, in some embodiments, it is done via one or more couplers (e.g., one or more nails, screws, bolts, staples, eyelets, magnets, adhesives, welds, interference fits, friction fits, tongue-and-groove connections, snaps, ties, rivets, stakes, wire ties, catches, frictional engagements, mechanical engagements, or any other suitable couplers), and in some embodiments the reinforcement elements are integrally formed with the endplates or other components of the implant (e.g., via additive manufacturing, 3D-printing, molding, etching, subtractive manufacturing, or in any other suitable manner). In some cases, a flexure assembly 110 that can be printed with additive manufacturing includes one or more novel shapes, such as support structures while the material is cooling so that the geometry does not sag, warp, or otherwise deform. In some embodiments, electropolishing is used, for instance, to improve fatigue performance or for any other suitable purposes. In some cases, however, the chemicals used in electropolishing can be cytotoxic. Accordingly, some embodiments of the described systems and methods include a flexure geometry that allows for post-polish cleaning (e.g., to allow all such chemicals to be removed). Indeed, in some cases, blind cavities, small voids, and other difficult to clean geometries are avoided.

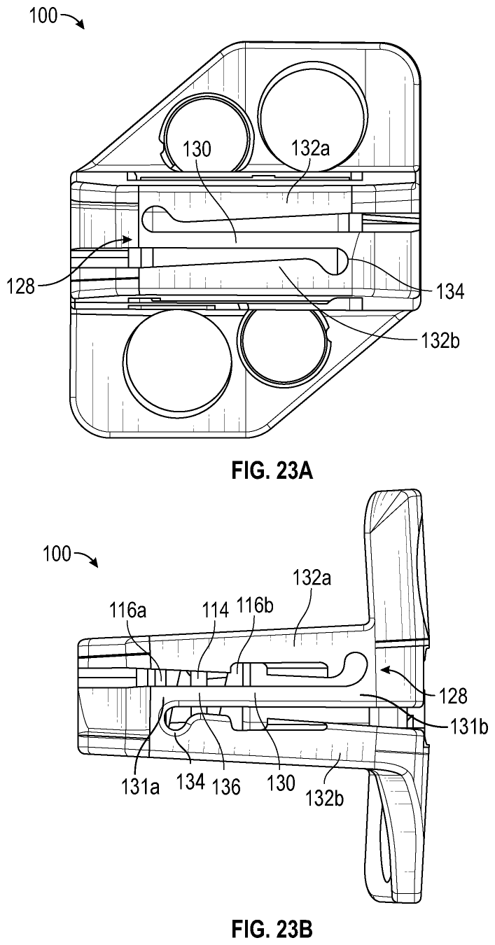

According to some embodiments, the reinforcement element 122 includes one or more springs 128. For example, some embodiments include one or more coil springs, as described above, and some embodiments include one or more leaf springs or other types of springs. In this regard, some embodiments include one or more resilient members 130, which in some cases are supported by one or more spring supports 132. In this regard, the resilient member can have any suitable characteristic of a flexure (e.g., allowing it to bend, rotate, cantilever, compress, place tension on one or more elements, flex, or otherwise modulate the kinematic profile of the implant 100). Similarly, the spring supports can have any suitable characteristic of the supports 116 of the flexure assembly 110. That said, in some cases, the resilient member is oriented perpendicular (or substantially perpendicular, such as within 1 degree, 2 degrees, 3 degrees, 5 degrees, 10 degrees, or 15 degrees of perpendicular) to the implant's axis of compression, such that the spring 128 is configured to resist compression of the implant. By way of non-limiting illustration, FIGS. 23A-23B show some embodiments of implants 100 with springs 128 having resilient members 130 supported by spring supports 132a, 132b, the springs 128 thereby being similar to a flexure assembly, but being rotated approximately 90 degrees (±up to 25 degrees (e.g., ±3 degrees, 5 degrees, 10 degrees, 15 degrees, or otherwise)) from the axis of compression, such that they modify the compression profile of the implant 100.

In some embodiments with a spring 128 having a resilient member 130, the resilient member is positioned in a particular position with respect to one or more flexures 114. In this regard, according to a pseudo rigid body model of physics, some embodiments of the resilient member include a particular pivot region or location (where the resilient member naturally pivots based on how it is coupled to the spring supports 132). In some cases, the pivot region is located at approximately 7.5% of the resilient member's length away from an end 131a or 131b of the resilient member (e.g., between 3% and 15%, between 5% and 10%, or otherwise around 7.5% from the end). In some embodiments, the implant 100 is structured such that the pivot region aligns with one or more flexures (e.g., such that there is a cross-point 136 where the flexure and the resilient member cross, and the cross point is located at the pivot region). In some embodiments, this alignment places minimal stress on the resilient member and the flexure (and other components of the implant). It should be noted that in some cases, the spring has one or more of a substantially consistent thickness and a substantially consistent material composition. That said, in some cases, the thickness of the spring (e.g., the resilient member) changes (e.g., increases, reduces, fluctuates, modulates, or otherwise changes) along its length. In some such cases, the pivot region moves (so in some cases, the cross point is disposed at a different location to align with the shifted pivot region).

By way of non-limiting illustration, FIG. 23B shows an implant 100 having a flexure 114 supported by a first support 116a and a second support 116b, as well as a spring 128 with a resilient member 130 supported by a first spring support 132a and a second spring support 132b, the resilient member 130 and the flexure 114 being substantially perpendicular to each other, and crossing each other at a cross point 136, with the cross point being disposed at the resilient member's 130 pivot region (e.g., about 7.5% of the resilient member's 130 length away from the first end 131a).

In some embodiments, the implant 100 includes one or more reliefs 134. Where a relief is included, the relief can include any geometric shape configured to reduce or spread out the stress placed on one or more components (or parts thereof). For example, in some cases, the relief includes one or more rounded portions, gaps, slots, undercuts, tear-drops, obround reliefs, eccentric reliefs, notches, or any other mechanical reliefs for reducing stress. In some cases, one or more reliefs is included in one or more springs 128, flexure assemblies 110, endplates 102, or other portions of the implant. By way of non-limiting illustration, FIG. 23B shows some embodiments of a spring 128 with a relief 134 in the form of a rounded portion configured to spread forces out along a juncture of a resilient member 130 and a spring support 132b.

Some embodiments of the implant 100 define one or more cavities 124. The cavity can include any suitable aperture, passage, recess, indentation, space, orifice, or other cavity configured to receive a material, such as a biological packing material. Where a biological material for packing into the implant is used, it can include any suitable biological material, such as one or more of ground bone, calcium phosphate, bone graft filler, or other packing material used in connection with spinal surgeries. In some cases, the material is granular, and in some cases the granules are sized so as to resist dissociation from the implant in large quantities (e.g., the granules are larger than the “pore size” of the gaps between coils of reinforcement members 122).

In some embodiments, one or more of the endplates 102, flexure assemblies 110, or other components of the implant 100 optionally include (or define) one or more windows 126 through which a cavity 124 can be accessed (e.g., through which biological material can be inserted into the cavity). Where a window is included, the window can be any suitable size (e.g., between 10% and 80% of the size of surface area of an endplate 102, or any subrange thereof) and any suitable shape (e.g., circular, semi-circular, square, rectangular, triangular, pentagonal, hexagonal, trapezoidal, polygonal, symmetrical, asymmetrical, or otherwise regularly or irregularly shaped). In some embodiments, all or part of the window is defined by one or more components of the implant, such as one or more portions of an endplate, one or more loading regions 112 of a flexure assembly 110, one or more reinforcement components 122, or any other components of the implant. By way of non-limiting illustration, FIG. 9 shows an embodiment of a window 124 formed via an L-shaped portion of a first endplate 102a and a first loading region 112a of a second flexure assembly 110b, the window being substantially rectangular (with rounded edges or corners) and having a size of less than 25%±15% of the external surface area of the first endplate, the window providing access to a cavity 124 disposed between the first endplate and a second endplate 102b, and the cavity being configured to receive a biological packing material.

According to some embodiments of the disclosed systems and methods, a method of providing the spinal implant 100 is provided. In some embodiments, the method includes forming the spinal implant. The spinal implant can be formed in any suitable manner, such as through casting (e.g., continuous casting, die casting, mold casting, resin casting, sand casting, or any other suitable type of casting), molding (e.g., metallurgy, compaction, injection molding, or any other suitable type of molding), forming (e.g., forging, extrusion, pressing, or any other suitable type of forming), machining (e.g., milling, turning, drilling, or any other suitable type of machining), joining (e.g., welding, ultrasonic welding, or any other suitable type of joining), additive manufacturing (e.g., 3D printing, deposition, layered manufacturing, or other additive manufacturing), subtractive manufacturing, suitable type of, or in any other suitable manner. For example, some embodiments of the implant are formed via additive manufacturing (e.g., 3D printing) in a manner that allows multiple (or all) components of the implant to be formed as a single integral part. That said, in some embodiments, one or more components are formed separately and coupled to one or more other components of the implant. In such cases, such coupling can be done in any suitable manner, such as via one or more anchors, adhesives, welds, magnets, interference fits, friction fits, mechanical engagements, tongue-and-groove connections, snaps, ties, rivets, stakes, wire ties, pins, catches, clamps, threaded engagements, or any other suitable couplers.

According to some embodiments, the method includes forming one or more components of the implant 100 to specific specifications (e.g., as discussed above). For example, in some embodiments, one or more components are formed to meet a certain threshold of flexibility, stability, stiffness, or any other suitable attribute.

In some embodiments, the method includes identifying desired parameters for the implant 100 to satisfy. Such parameters can include any suitable parameters, such as flexibility (either in general or along one or more specific axes), stiffness, size, shape, or other aspects of the implant. In some cases, the desired parameters are patient-specific, spinal-segment-specific, or otherwise dependent on the desired use case. Moreover, such parameters can be identified based on any suitable information, such as patient demographics, imaging, patient symptoms (or other patient-provided information), test results, bone density information, bone shape information, or any other suitable information. By way of non-limiting illustration, some embodiments of the method include performing pre-operative imaging to identify desired parameters.

Where the method includes pre-operative imaging, the imaging can include any suitable type of imaging, such as X-ray, CT Scan, MRI (or any variant thereof, such as fMRI), ultrasound, or any other imaging technique. By way of non-limiting illustration, in some embodiments, the parameters are determined based on water content as measured via MRI, native flexibility as measured via functional X-rays, or other information obtained through imaging. In this regard, unlike many (if not all competing systems and methods), some embodiments of the described systems and methods include matching the index level to the adjacent levels in terms of radiographic height, stiffness, and range of motion. In this regard, using a simple series of flexion/extension X-rays add little to no additional burden in pre-operative imaging.

Some embodiments of the implant 100 are configured to be custom tailored to a patient's needs. Accordingly, some embodiments of the method include tailoring the implant to a patient's needs. Such tailoring can be done in any suitable manner, such as prior to (or during) formation of the implant or any of its constituent components, or by making adjustments after the formation of the implant. In some embodiments where adjustments are made after the formation of the device, such adjustments can include any suitable adjustments to the dimensions, shape, orientation, or other configuration of any of the components. For example, suitable adjustments can include: adding or removing material (e.g., to make one or more flexure 114 more or less stiff, removing one or more reinforcement elements 122, etc.); adjusting the position of one or more components (e.g., rotating a flexure assembly 110 to adjust the flexibility along certain axes); reshaping one or more components (e.g., to provide a more even load path through one or more flexure assemblies); increasing or decreasing the stiffness of a support 116, a flexure, a coupler 118, or another component (e.g., by changing the length, width, or height of such component, or by changing its material composition, or by changing its general shape or configuration); limiting flexibility through a stop element (e.g., a contact-enabled stop); or otherwise adjusting one or more components or parameters of the implant. In any case, it should be noted that the size and shape of primary fixation does not necessarily preclude multiple level implantations.

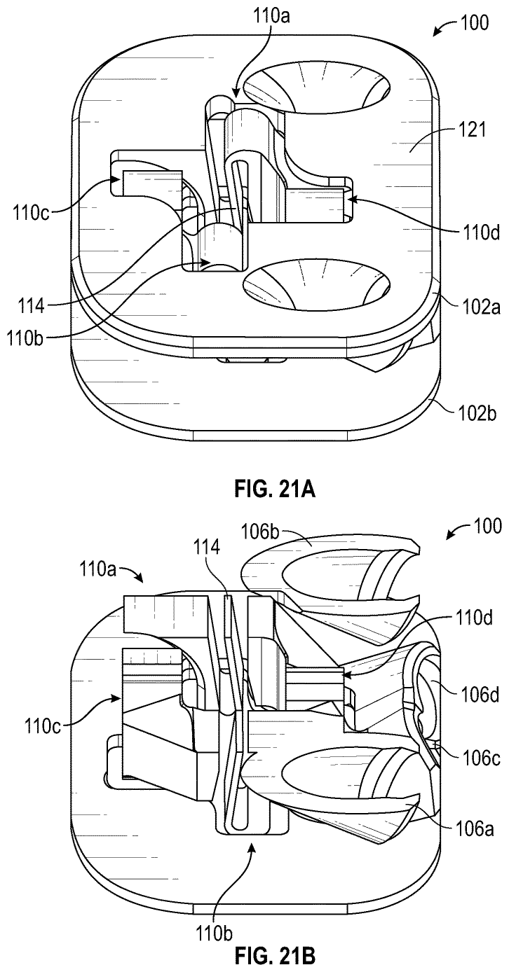

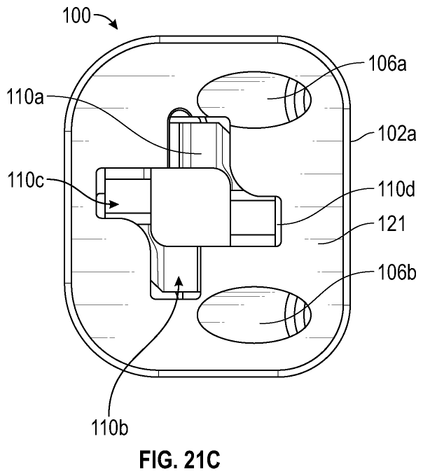

The systems and methods discussed herein can be modified in any suitable manner. To illustrate, some embodiments include multiple flexure assemblies 110 having a specific configuration. For example, the flexure assemblies of some embodiments are generally arranged in a X shape, an N shape, a Z shape, an S shape, a T shape, a plus shape, a cross shape, an H shape, a V shape, a W shape, or any other suitable configuration that allows the implant 100 to function as described herein. In this regard, an X shape or another shape with one or more centrally concentrated components can be particularly useful in some cases, because resistance to torsion may be reduced through minimization of outer fibers. In this regard, torsion resistance can, in some embodiments, be stronger when there is more material disposed farther away from an axis of rotation (e.g., a rod with a large diameter is more difficult to twist than is a rod with a small diameter, even if the amount of material used in both rods is the same). By way of non-limiting illustration, FIG. 13 shows an embodiment of the implant 100 with an X-shaped arrangement of flexure assemblies 110a, 110b, 110c, and 110d, thereby reducing torsional stiffness. FIGS. 21A-21C provide another non-limiting illustration of some embodiments of an implant 100 that includes four flexure assemblies 110a, 110b, 110c, and 110d, arranged generally in a cross or plus shaped pattern, but with each flexure assembly being spatially separated from the others such that each can operate independently (while still contributing its flexibility attributes to the overall whole). In FIGS. 21A-21C, flexure assemblies 110a and 110b are configured to contribute mainly to the lateral bending attributes of the implant 100, while flexure assemblies 110c and 110d are configured to contribute mainly to flexion/extension.

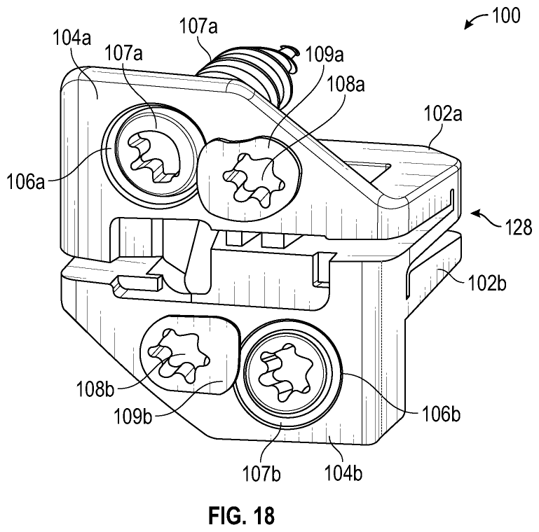

Additionally, some embodiments use compression-based flexure assemblies 110 (instead of or in addition to tension-based flexure assemblies). However, instead of thick, bulky, trunk-like compression-based stems 54, some embodiments utilize elongated flexures 114 configured to run largely parallel (e.g., within 30 degrees, 25 degrees, 20 degrees, 15 degrees, 10 degrees, 5 degrees, 3 degrees, or 2 degrees of parallel) with the endplates 102 (or largely perpendicular to the direction of compression) to prevent buckling. In some embodiments, flexure assemblies include grooves or contours that extend from the flexure to one or more components or surfaces of the implant 100. By way of non-limiting illustration, FIG. 18 shows an implant 100 having a compression-based flexure assembly 110 disposed between a first endplate 102a and a second endplate 102b, with such flexure assembly having a flexure oriented largely parallel to the endplates.

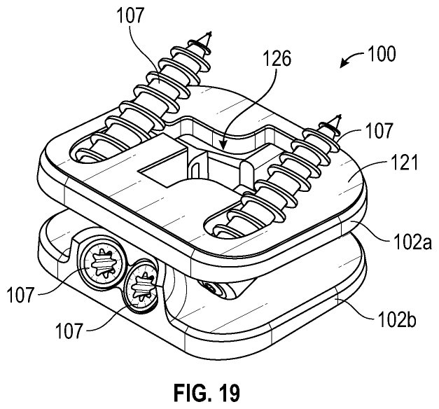

According to some embodiments, multiple primary fixation passages 106 are included at each endplate 102. Indeed, in some cases, the implant 100 includes a first primary fixation passage 106a, a second primary fixation passage 106b, a third primary fixation passage 106c, a fourth primary fixation passage 106d, and any number of additional primary fixation passages. In this regard, the primary fixation passages can have any position or orientation with respect to each other or with respect to the other components of the implant, provided they allow for anchors 107 to be inserted therethrough and into a suitable location of the patient (e.g., a bone of a spinal vertebra). In some cases, primary fixation passages are formed through the endplates themselves as opposed to being formed through one or more flanges 104. By way of non-limiting illustration, FIGS. 19-22B show implants 100 having primary fixation passages 106a, 106b, 106c, 106d formed through the endplates 102a, 102b. In these FIGS., there are two primary fixation passages formed through the top endplate 102a and two formed through the bottom endplate 102b, where the ones formed through the top endplate are placed more laterally than the two formed through the bottom endplate. With this configuration, a practitioner can access the drives of the anchors 107 placed through the passages. As shown in FIG. 19, some configurations are designed to allow for multiple anchors to enter a single bony surface (and in some cases, this is done while still allowing access to (e.g., without occluding) a window 126 or another feature of the implant 100).

According to some embodiments, the flexures 114 of multiple flexure assemblies 110 are configured to cross. For example, in some cases, a first end (e.g., a top) of a first flexure is ventral to a first end (e.g., a top) of a second flexure, whereas a second end (e.g., a bottom) of the first flexure is dorsal to a second end (e.g., a bottom) of the second flexure (or the ends are lateral to each other, whereas the other ends are inversely lateral to each other, the ends switch cranially-caudally, or the flexures are otherwise configured to cross). In this regard, crossed flexures can (in some embodiments) add strength to the implant 100 as a whole, while still maintaining its instantaneous axis of rotation (IAR) characteristics and capabilities. By way of non-limiting illustration, FIGS. 20A and 20B show some embodiments of implants 100 with flexure assemblies 110 having flexures 114 that are configured to cross.

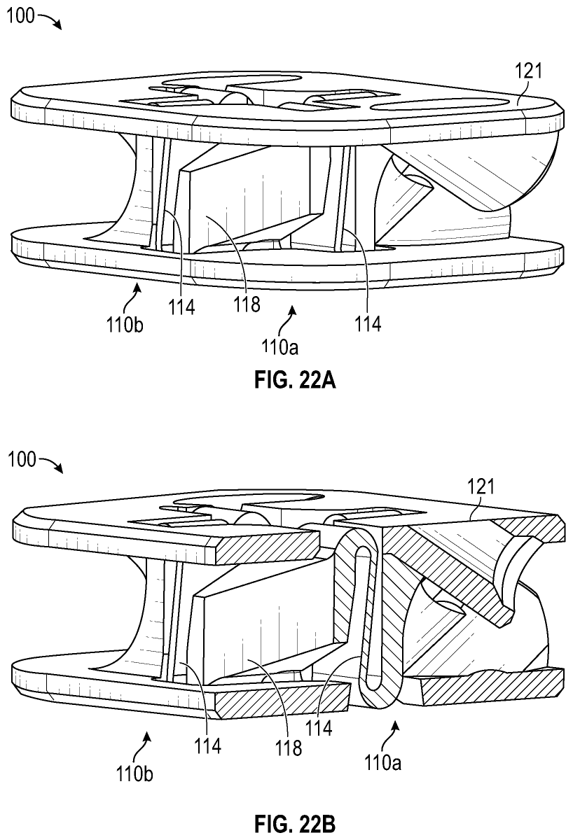

As discussed above, some embodiments with multiple flexure assemblies 110 include one or more couplers 118 configured to link multiple flexure assemblies together. In some cases, such couplers themselves operate as flexures or as other flexible elements configured to impart strength, resilience, flexibility, or any other desirable and suitable attributes to the implant 100. Thus, such couplers can have any shape or configuration that allows them to couple two or more flexure assemblies together in a desirable manner. By way of non-limiting illustration, FIGS. 22A-22B each show an implant 100 having a first flexure assembly 110a and a second flexure assembly 110b, where such flexure assemblies are joined together by a coupler 118, and where the coupler is generally cuneate (or wedge-shaped), tapering from a thicker portion to a thinner portion. In saying this, it is noted that the coupler can (in some embodiments) have any other suitable shape.

As discussed above, some embodiments include one or more porous structures 120 configured to help stimulate bone growth. In some embodiments, one or more surfacing materials 121 is used with or instead of the porous structure. Where a surfacing material is used, the surfacing material can include any suitable porous, rough, contoured, ridged, grooved, wavy, undulated, spongey, porous, bumpy, uneven, knurled, latticed, powder coated, sintered, anodized, electroplated, ground, buffed, etched, or otherwise composed or treated material configured to stimulate bone growth. In some cases, such material is disposed on one or more of the endplates 102 or any other suitable surface or portion of the implant 100. By way of non-limiting illustration, FIGS. 19, 21A, 21C, 22A, and 22B each show an embodiment of an implant 100 having a surfacing material 121 disposed on a cranial surface and a caudal surface (e.g., a first endplate 102 and a second endplate 102b), although the surfacing material is only visible on the cranial surface in these figures, as the caudal surfaces are obscured.

Any and all of the components in the figures, embodiments, implementations, instances, cases, methods, applications, iterations, examples, elements, and other parts of this disclosure and the implant 100 can be combined in any suitable manner. Additionally, any component can be removed, separated from other components, modified with or without modification of like components, or otherwise altered together or separately from anything else disclosed herein.

As used herein, the singular forms “a”, “an”, “the” and other singular references include plural referents, and plural references include the singular, unless the context clearly dictates otherwise. For example, reference to a flexure (or “the” flexure) includes reference to one or more flexures, and reference to supports includes reference to one or more supports. In addition, where reference is made to a list of elements (e.g., elements a, b, and c), such reference is intended to include any one of the listed elements by itself, any combination of less than all of the listed elements, and/or a combination of all of the listed elements. Moreover, the term “or” by itself is not exclusive (and therefore may be interpreted to mean “and/or”) unless the context clearly dictates otherwise. Similarly, the term “and” by itself is not exclusive (and therefore may be interpreted to mean “and/or”) unless the context clearly dictates otherwise. Furthermore, the terms “including”, “having”, “such as”, “for example”, “e.g.”, and any similar terms are not intended to limit the disclosure, and may be interpreted as being followed by the words “without limitation”.

In addition, as the terms “on”, “disposed on”, “attached to”, “connected to”, “coupled to”, etc. are used herein, one object (e.g., a material, element, structure, member, etc.) can be on, disposed on, attached to, connected to, or otherwise coupled to another object—regardless of whether the one object is directly on, attached, connected, or coupled to the other object, or whether there are one or more intervening objects between the one object and the other object. Also, directions (e.g., “front”, “back”, “on top of”, “below”, “above”, “top”, “bottom”, “side”, “up”, “down”, “under”, “over”, “upper”, “lower”, “lateral”, “right-side”, “left-side”, “base”, etc.), if provided, are relative and provided solely by way of example and for ease of illustration and discussion and not by way of limitation.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, examples, and illustrations set forth herein can be combined in any suitable manner with any other components or characteristics from one or more other embodiments, examples, and illustrations described herein.

What is claimed is:

1. An implant for standalone interbody fusion, the implant comprising:
a first endplate;
a second endplate; and
a load-bearing component disposed between the first endplate and the second endplate,
wherein the load-bearing component has a height that is at least twice as great as at least one of a width and a length of the load-bearing component.

2. The implant of claim 1, wherein at least one of the first endplate and the second endplate comprises a flange, the flange comprising a primary fixation passage configured to receive an anchor for coupling at least one of the first endplate and the second endplate to a spinal segment of a patient.

3. The implant of claim 2, wherein the flange further comprises a secondary fixation passage configured to receive a locking cap, wherein the locking cap is configured to help prevent the anchor from backing out of the primary fixation passage.

4. The implant of claim 1, wherein the load-bearing component comprises a first flexure, and wherein a portion of the first flexure is configured to be placed under tension when a compressive force is applied to at least one of the first endplate and the second endplate.

5. The implant of claim 4, further comprising a flexure assembly comprising:
a first loading region configured to receive a first portion of the compressive force;
a second loading region configured to receive a second portion of the compressive force;
the first flexure;
a first support configured to transfer the first portion of the compressive force to a first end of the first flexure,
wherein the first end of the first flexure is positioned opposite the first loading region; and
a second support configured to transfer the second portion of the compressive force to a second end of the first flexure,
wherein the second end of the first flexure is positioned opposite the second loading region, and
wherein a transfer of the compressive force from the first loading region and the second loading region causes the first flexure to support the compressive force with an opposing tensile force.

6. The implant of claim 5, further comprising a second flexure assembly, wherein the second flexure assembly comprises a second flexure.

7. The implant of claim 6, wherein the first flexure is configured to be flexible along a flexion/extension axis, and wherein the second flexure is configured to be flexible along a lateral bending axis.

8. The implant of claim 1, wherein the implant is flexible along each of a flexion/extension axis, a lateral bending axis, and a torsion axis.

9. The implant of claim 8, wherein the implant is configured to stabilize motion to a physiological range along each of the flexion/extension axis, the lateral bending axis, and the torsion axis.

10. The implant of claim 1, wherein the load-bearing component is configured to withstand at least 25% of a compression force applied to the first endplate and the second endplate without buckling and without breaking when the compression force results from compression of the implant between a first vertebra and a second vertebra of a patient's spine.

11. The implant of claim 1, further comprising a reinforcement element configured to further stabilize the implant.

12. The implant of claim 11, wherein the reinforcement element is included in an array of reinforcement elements forming a porous structure.

13. A flexure-based standalone interbody fusion implant comprising:
a first flexure assembly comprising:
a first loading region configured to receive a first portion of a compressive force;
a second loading region configured to receive a second portion of the compressive force;
a first support associated with the first loading region;
a second support associated with the second loading region; and
a flexure coupled to the first support and the second support,
wherein a portion of the flexure is configured to be placed under tension in response to the compressive force being applied to at least one of the first loading region and the second loading region.

14. The flexure-based standalone interbody fusion implant of claim 13, wherein at least one of a stiffness of the implant along a flexion/extension axis, a stiffness of the implant along a lateral bending axis, and a stiffness of the implant along a torsion axis is selectively adjustable.

15. The flexure-based standalone interbody fusion implant of claim 13, wherein a flexibility of the implant along a flexion/extension axis is 7 degrees±4 degrees, a flexibility of the implant along a lateral bending axis is 5 degrees±3 degrees, and a flexibility of the implant along a torsion axis is 4 degrees±4 degrees.

16. The flexure-based standalone interbody fusion implant of claim 13, further comprising a second flexure assembly.

17. The flexure-based standalone interbody fusion implant of claim 16, wherein the second flexure assembly is coupled to the first flexure assembly via a coupler.

18. The flexure-based standalone interbody fusion implant of claim 16, wherein the first flexure assembly has a first orientation and the second flexure assembly has a second orientation, and wherein the first orientation is different from the second orientation.

19. A method of providing an implant, the method comprising:
obtaining a first flexure assembly comprising:
a first loading region configured to receive a first portion of a compressive force;
a second loading region configured to receive a second portion of the compressive force;
a first support associated with the first loading region;
a second support associated with the second loading region; and
a flexure coupled to the first support and the second support, wherein the flexure is configured to be placed under tension in response to the compressive force being applied to at least one of the first loading region and the second loading region; and
inserting the implant into an interbody space of a spine of a patient such that adjacent vertebrae of the spine apply the compressive force to the implant.

20. The method of claim 19, further comprising configuring at least one of the first support, the second support, and the flexure to provide a patient-specific flexibility along at least one of a flexion/extension axis, a lateral bending axis, and a torsion axis.
